(19) United States
(12) Patent Application Publication
(10) Pub. No.: US 2024/0197426 A1
(43) Pub. Date: Jun. 20, 2024

(54) MAGNETICALLY COUPLED ABLATION COMPONENTS

(71) Applicant: AtriCure, Inc., Mason, OH (US)

(72) Inventors: Matthew Monti, Cincinnati, OH (US); Kenneth Lance Miller, Hamilton, OH (US); Keith Edward Martin, Dayton, OH (US); Salvatore Privitera, Mason, OH (US); Tamer Ibrahim, Danville, CA (US); Jeremy D. Dando, Plymouth, MN (US); Adam Wesley Cates, Minneapolis, MN (US); Paighton Anne Schrupp, St. Louis Park, MN (US); Sonja Kosir, Cincinnati, OH (US); Liam Douglas Malone, Chatteris (GB); Joseph Thomas Batley, Impington (GB)

(73) Assignee: AtriCure, Inc., Mason, OH (US)

(21) Appl. No.: 18/545,190

(22) Filed: Dec. 19, 2023

Related U.S. Application Data
(60) Provisional application No. 63/433,960, filed on Dec. 20, 2022.
(60) Provisional application No. 63/433,963, filed on Dec. 20, 2022.

(51) Int. Cl. A61B 34/00; A61B 18/00

(52) U.S. Cl. CPC A61B 34/73; A61B 18/00; A61B 2017/00292

(57) ABSTRACT

An ablation system and method provides for cooperatively transdermally ablating cardiac tissue by cooperating epicardial and endocardial components. The ablation system includes a first ablation component configured to be positioned on a first side of a target tissue. The first ablation component includes a first ablation element and a first magnetic element. The ablation system includes a second ablation component configured to be positioned on a second side of a target tissue. The second side is opposite the first side. The second ablation component includes a second ablation element and a second magnetic element. The first magnetic element and the second magnetic element are configured to cooperate to facilitate positioning at least one of the first ablation component and the second component on the target tissue. The first ablation element and the second ablation element are configured to cooperate to create a lesion in the target tissue.

### CROSS-REFERENCE TO RELATED APPLICATIONS

The present application claims the benefit of two U.S. provisional patent applications Ser. Nos. 63/433,960 and 63/433,963, both entitled “Magnetically Coupled Ablation Components”, and both filed on 20 Dec. 2022, the disclosures of which are hereby incorporated by reference in their entirety.

### TECHNICAL FIELD

The present application is directed to medical instruments and devices and related methods, and, more specifically, to devices for ablating tissue and related methods. Those skilled in the art, however, will understand that the present disclosure may be applicable to other surgical procedures beyond tissue ablation.

### BACKGROUND OF THE INVENTION

In the context of cardiac ablation, catheter-based ablation devices may be used for accessing the endocardial (interior) surface of the heart, but may not have easy access to the epicardial (exterior) surface of the heart. Surgical-based ablation devices may easily access the epicardial surface, either through open-chest procedures or minimally invasive procedures, but may require atriotomies to directly access the endocardial surface. Some devices may use vacuum to fold tissue so that the endocardial surfaces are between electrodes, but these devices may be limited by the size and/or thickness of tissue that can be engaged. Radio frequency (RF) clamp devices may include electrodes on opposing jaws and can be used epicardially by pinching together two walls or with one jaw endocardial and the opposing jaw epicardial.

Problems encountered in cardiac ablation procedures may include difficulty in precisely locating ablation apparatus on the target tissue and difficulties related to the ablation of the tissue. For example, in some circumstances, it may be difficult to create a sufficiently continuous, sufficiently deep (e.g., transmural) line of ablated tissue, which may be necessary to electrically isolate a portion of the heart. While known devices have been used safely and effectively to ablate tissue (such as cardiac tissue), improvements in the construction and operation of surgical devices for ablating tissue may be beneficial for users (e.g., surgeons) and patients.

### SUMMARY OF THE INVENTION

The present disclosure includes various improvements which may enhance the construction, operation, and methods of use of devices for ablating tissue, such as cardiac tissue.

It is a first aspect of the present disclosure to provide an ablation system comprising: (i) a first ablation component configured to be positioned on a first side of a target tissue, the first ablation component comprising a first ablation element and a first magnetic element; (ii) a second ablation component configured to be positioned on a second side of a target tissue, the second side being opposite the first side, the second ablation component comprising a second ablation element and a second magnetic element, where the first magnetic element and the second magnetic element are configured to cooperate to facilitate positioning at least one of the first ablation component and the second component on the target tissue, and where the first ablation element and the second ablation element are configured to cooperate to create a lesion in the target tissue.

In a more detailed example of the first aspect, one or more of the first magnetic element or the second magnetic element comprises one or more of a permanent magnet, an electromagnet, or a ferromagnetic material. In yet another more detailed example, one or more of the first ablation element or the second ablation element is configured for one or more of radiofrequency (RF) energy ablation, pulsed field ablation, cryoablation, ultrasound ablation, or laser ablation. In a further detailed example, one or more of the first magnetic element or the second magnetic element comprises a plurality of magnetic elements. In still a further detailed example, one or more of the first ablation element or the second ablation element comprises a plurality of ablation elements. In a more detailed example, the first ablation component and the second ablation element comprise selectively engageable cooperating surface features configured to engage the target tissue therebetween. In a more detailed example, one of the first ablation component or the second ablation component comprises a tissue anchor, the tissue anchor being configured to secure the one of the first ablation component or the second ablation component to the target tissue.

It is a second aspect of the present disclosure to provide a method of creating a lesion, the method comprising: (i) placing a first ablation component on a first surface of a target tissue, the first ablation component comprising a first magnetic element and a first ablation element; (ii) placing a second ablation component on a second surface of a target tissue, the second ablation component comprising a second magnetic element and a second ablation element; (iii) positioning one or more of the first ablation component or the second ablation component using magnetic cooperation between the first magnetic element and the second magnetic element; and (iv) creating a first lesion in the target tissue using the first ablation element and the second ablation element.

In a more detailed example of the second aspect, one or more of the first magnetic element or the second magnetic element comprises an electromagnet, and positioning the one or more of the first ablation component or the second ablation component using magnetic cooperation between the first magnetic element and the second magnetic element comprises energizing the electromagnet. In yet another more detailed example, creating the lesion in the target tissue using the first ablation element and the second ablation element comprises one or more of radiofrequency (RF) energy ablation, pulsed field ablation, cryoablation, ultrasound ablation, or laser ablation. In a further detailed example, the method further includes repositioning the one or more of the first ablation component or the second ablation component, and creating a second lesion in the target tissue proximate the first lesion via at least one of simultaneous monopolar ablation and bipolar ablation. In still a further detailed example, the method further includes securing one or more of the first ablation component or the second ablation component to the target tissue using a tissue anchor.

It is a third aspect of the present invention to provide an ablation system comprising: (i) a first ablation component configured to be positioned on a first side of a target tissue, the first ablation component comprising a first magnetic element; (ii) a second ablation component configured to be positioned on a second side of a target tissue, the second side being opposite the first side, the second ablation component comprising a second magnetic element, where the first magnetic element and the second magnetic element are configured to cooperate to facilitate positioning at least one of the first ablation component and the second ablation component on the target tissue.

In a more detailed example of the third aspect, one or more of the first magnetic dement or the second magnetic element comprises one or more of a permanent magnet an electromagnet or a ferromagnetic material. In yet another more detailed example, one or more of the first magnetic element or the second magnetic element comprises a plurality of magnetic elements. In a further detailed example, one or more of the first magnetic element or the second magnetic clement comprises an electromagnet, and where the first magnetic element and the second magnetic element are configured to cooperate to facilitate positioning at least one of the first ablation component and the second component on the target tissue by energizing the electromagnet. In still a further detailed example, one or more of the first magnetic element or the second magnetic element is generally in the form of a cuboid, a cylinder, a horseshoe, a cone. In a more detailed example, one or more of the first magnetic element or the second magnetic element comprises an electromagnet comprising two or more selectively energizable coils. In a more detailed example, one or more of the first magnetic element or the second magnetic element is configured to control a magnetic clamping force between the first ablation component and the second ablation component.

It is a fourth aspect of the present disclosure to provide a method of creating a lesion, the method comprising: (i) placing a first ablation component on a first surface of a target tissue, the first ablation component comprising a first ablation magnetic element; (ii) placing a second ablation component on a second surface of a target tissue, the second ablation component comprising a second ablation magnetic element; (iii) aligning and compressing the target tissue between the first and second ablation magnetic elements using magnetic attraction; and, (iv) creating a first lesion in the target tissue using at least one of the first ablation magnetic element and the second ablation magnetic element.

In a more detailed example of the fourth aspect, at least one of the first ablation magnetic element and the second ablation magnetic element comprises an electromagnet, and aligning and compressing the target tissue between the first and second ablation magnetic elements using magnetic attraction includes energizing the electromagnet. In yet another more detailed example, creating the first lesion in the target tissue using at least one of the first ablation magnetic element and the second ablation magnetic element includes using at least one of radiofrequency (RF) energy ablation, pulsed field ablation, cryoablation, ultrasound ablation, and laser ablation. In a further detailed example, the method further includes repositioning at least one of the first ablation magnetic component and the second ablation magnetic component, and creating a second lesion in the target tissue proximate the first lesion. In still a further detailed example, the method further includes securing at least one of the first ablation magnetic component and the second ablation magnetic component to the target tissue using a tissue anchor.

It is a fifth aspect of the present disclosure to provide an ablation device comprising: (i) a first ablation component configured to be positioned on a first side of a target tissue, the first ablation component comprising a first ablation element and a first magnetic element; (ii) a first catheter operatively coupled to the first ablation component; and, (iii) a second catheter having a through conduit sized to receive the first ablation component and the first catheter.

In yet another more detailed example of the fifth aspect, the first ablation element and the first magnetic element are synonymous. In yet another more detailed example, the ablation device further includes an atraumatic distal tip. In a further detailed example, the atraumatic distal tip comprises at least one of the first ablation element and the first magnetic element. In still a further detailed example, at least one of the first ablation element and the first magnetic element is proximal to the distal tip. In a more detailed example, both the first ablation element and the first magnetic element are proximal to the distal tip. In a more detailed example, the first ablation element is distal with respect to the first magnetic element. In another more detailed example, a longitudinal distance separates the first ablation element with respect to the first magnetic element. In yet another more detailed example, the longitudinal distance is created by at least one of a spacer and a longitudinal gap. In still another more detailed example, the ablation device further includes a second magnetic element, where the first ablation element interposes the first and second magnetic elements.

In a more detailed example of the fifth aspect, a first longitudinal distance separates the first ablation element with respect to the first magnetic element, and a second longitudinal space separates the first ablation element with respect to the second magnetic element. In yet another more detailed example, the first and second longitudinal distances are created by at least one of a spacer and a longitudinal gap. In a further detailed example, the first magnetic element is positioned within the first ablation element. In still a further detailed example, the first ablation element is proximate a distal end of the first ablation component. In a more detailed example, the first ablation element is stationary and the first magnetic element is repositionable along a path of the first ablation element. In a more detailed example, the first ablation element includes a plurality of first ablation elements, and wherein the first magnetic element is repositionable along a path overlapping at least one of the plurality of first ablation elements. In another more detailed example, the ablation device further includes a second ablation component configured to be positioned on the first side of the target tissue, the second ablation component comprising a second ablation element and a second magnetic element.

In yet another more detailed example of the fifth aspect, the second ablation element includes a plurality of second ablation elements, and wherein the second magnetic element is repositionable along a path overlapping at least one of the plurality of second ablation elements. In yet another more detailed example, the ablation device further includes a third ablation component configured to be positioned on the first side of the target tissue, the third ablation component comprising a third ablation element and a third magnetic element. In a further detailed example, the third ablation element includes a plurality of third ablation elements, and wherein the third magnetic element is repositionable along a path overlapping at least one of the plurality of third ablation elements. In still a further detailed example, the first magnetic element is stationary and the first ablation element is repositionable along a path of the first magnetic element. In a more detailed example, the first ablation component resides on a flexible pad proximate a distal end of the ablation device. In a more detailed example, the flexible pad is configured to be folded and unfolded to change its shape. In another more detailed example, the flexible pad is configured to be at least one of curled and wound to change its shape. In yet another more detailed example, the first ablation component includes a connecting component configured to be operatively coupled to a control unit. In still another more detailed example, the first ablation component resides on a pad proximate a distal end of the ablation device, the pad including a plurality of first ablation elements and a plurality of first magnetic elements.

In a more detailed example of the fifth aspect, the pad includes an inflatable bladder. In yet another more detailed example, the plurality of first ablation elements and the plurality of first magnetic elements are operatively coupled to the pad. In a further detailed example, the plurality of first ablation elements and the plurality of first magnetic elements are at least one of printed on and embedded in the pad. In still a further detailed example, the plurality of first ablation elements and the plurality of first magnetic elements are mounted to the inflatable bladder. In a more detailed example, the plurality of first ablation elements and the plurality of first magnetic elements are at least one of printed on and embedded in the inflatable bladder. In a more detailed example, the first ablation element includes a plurality of first magnetic elements. In another more detailed example, the distal end of the ablation device includes an atraumatic distal tip, and the atraumatic distal tip is bulbous.

In yet another more detailed example of the fifth aspect, the atraumatic distal tip includes at least one of the first ablation element and the first magnetic element. In yet another more detailed example, the distal end of the ablation device includes an atraumatic distal tip, and the atraumatic distal tip includes at least one of a concavity, an opening, and a ring. In a further detailed example, the atraumatic distal tip includes at least one of the first ablation element and the first magnetic element. In still a further detailed example, the first magnetic element comprises at least one of a permanent magnet and an electromagnet. In a more detailed example, the first magnetic element includes an electromagnet core shaped as at least one of a cuboid, a cylinder, a horseshoe, a semi-circle, and the electromagnet core includes a first conductive winding wound therearound. In a more detailed example, the electromagnet core includes a second conductive winding wound therearound. In another more detailed example, the first conductive winding is wound around the electromagnet core along a first length, and the second conductive winding is wound around the electromagnet core along a second length, the first length being greater than the second length. In yet another more detailed example, the electromagnet core embodies a cylindrical shape, the first conductive winding is wound around the electromagnet core in a first direction, and the second conductive winding is wound around the electromagnet core in a second direction, generally opposite the first direction. In still another more detailed example, the first conductive winding is wound around the electromagnet core in a first direction, and the second conductive winding is wound around the electromagnet core in a second direction, generally opposite the first direction.

In a more detailed example of the fifth aspect, the first magnetic element includes a plurality of electromagnet cores, at least some of the plurality of electromagnet cores shaped as at least one of a cuboid, a cylinder, a horseshoe, and a semi-circle, and each of the plurality of electromagnet cores includes a first conductive winding wound therearound. In yet another more detailed example, each of the plurality of electromagnet cores includes a second conductive winding wound therearound. In a further detailed example, the first conductive winding is wound around each of the plurality of electromagnet cores in a first direction, and the second conductive winding is wound around each of the plurality of electromagnet cores in a second direction, generally opposite the first direction. In still a further detailed example, the plurality of electromagnet cores are arranged in a plurality of rows, and adjacent electromagnet cores are configured to have opposite polarities. In a more detailed example, the first magnetic element includes a permanent magnet. In a more detailed example, the first magnetic element includes a plurality of permanent magnets. In another more detailed example, the plurality of permanent magnets are arranged in a plurality of rows, and adjacent permanent magnets have opposite polarities.

In yet another more detailed example of the fifth aspect, the first magnetic element includes an electromagnet core embodying a horseshoe cross-section, and the electromagnet core includes a first conductive winding wound therearound. In yet another more detailed example, the first magnetic element includes a plurality of electromagnet cores operatively coupled to one another and repositionable between an expanded configuration and a retracted configuration, where the expanded configuration embodies increased spacing between the plurality of electromagnet cores and the retracted configuration embodies decreased spacing between the plurality of electromagnet cores, and each of the plurality of electromagnet cores includes a first conductive winding wound therearound. In a further detailed example, the expanded configuration embodies a first longitudinal cross-section having the plurality of electromagnetic cores distributed radially equidistant a first distance from a longitudinal axis, and the retracted configuration embodies a second longitudinal cross-section having the plurality of electromagnetic cores distributed radially equidistant a second distance from the longitudinal axis, the second distance being less than the first distance. In still a further detailed example, the first ablation component includes a sensor configured to detect at least one of a magnetic field and a change in a magnetic field. In a more detailed example, the sensor comprises at least one of a Hall Effect sensor, a magnetoresistive sensor, and a magnetic amplifier sensor. In a more detailed example, the first ablation component includes an expandible element. In another more detailed example, the expandible element includes an inflatable bladder. In yet another more detailed example, the expandible element is operatively coupled to at least one of the first ablation element and the first magnetic element. In still another more detailed example, the first magnetic element comprises a plurality of magnetic elements, and the expandible element is operatively coupled to the plurality of magnetic elements.

It is a sixth aspect of the present disclosure to provide a method of monitoring a tissue compression procedure, the method comprising: (i) positioning a first medical device and a second medical device so as to be interposed by tissue, the first medical device including a magnet, the second medical device including at least one of a magnet and a magnetically attractive material, where at least one of the first medical device and the second medical device includes a magnetic sensor; (ii) obtaining outputs from the magnetic sensor while the medical devices are interposed by the tissue; and, (iii) estimating at least one of a distance between the first medical device and the second medical device, and a compression force applied to the tissue by at least one of the first medical device and the second medical device.

It is a seventh aspect of the present disclosure to provide an ablation device comprising: (i) a first ablation component configured to be positioned on a first side of a target tissue, the first ablation component comprising a first ablation element and a first magnetic element; (ii) a first tether operatively coupled to the first ablation component; and, (iii) a catheter having a through conduit sized to receive the first ablation component and the tether.

In yet another more detailed example of the seventh aspect, the first ablation element and the first magnetic element are synonymous. In yet another more detailed example, the ablation device further includes an atraumatic distal tip. In a further detailed example, the atraumatic distal tip comprises at least one of the first ablation element and the first magnetic element.

These and other features are explained more fully in the examples illustrated below. It should be understood that in general the features of one example also may be used in combination with features of another example and that the examples are not intended to limit the scope of the invention.

### BRIEF DESCRIPTION OF DRAWINGS

The description of the examples described herein can be read with reference to the accompanying figures. It will be appreciated that for simplicity and clarity of illustration, elements illustrated in the figures have not necessarily been drawn to scale. For example, the dimensions of some of the elements are exaggerated relative to other elements. Examples incorporating teachings of the present disclosure are shown and described with respect to the figures presented herein, in which:

FIG. 1 is a simplified schematic view of a first example ablation system in use on a heart 10, according to at least some aspects of the present disclosure.

FIG. 2 is a perspective view of a second example ablation component, according to at least some aspects of the present disclosure.

FIG. 3 is a perspective view of a third example ablation component, according to at least some aspects of the present disclosure.

FIG. 4 is an elevation view of a fourth example ablation component 400 in two orientations, according to at least some aspects of the present disclosure.

FIG. 5 is a perspective view of a fifth example ablation component, according to at least some aspects of the present disclosure.

FIG. 6 is a perspective view of a sixth example ablation component, according to at least some aspects of the present disclosure.

FIG. 7 is a perspective view of a seventh example ablation component, according to at least some aspects of the present disclosure.

FIG. 8 is a perspective view of an eighth example ablation component, according to at least some aspects of the present disclosure.

FIG. 9A is a perspective view of a ninth example ablation component, according to at least some aspects of the present disclosure.

FIG. 9B is a detailed perspective view of a distal portion of the ablation component of FIG. 9A, according to at least some aspects of the present disclosure.

FIG. 10 is a simplified perspective view of an ablation component, according to at least some aspects of the present disclosure.

FIG. 11A is a detailed perspective view of a portion of an ablation component, according to at least some aspects of the present disclosure.

FIG. 11B is a perspective view of an ablation component, according to at least some aspects of the present disclosure.

FIG. 12 is a perspective view of an example of end-to-end coupling of ablation components, according to at least some aspects of the present disclosure.

FIG. 13 is a perspective view of an example of end-to-end coupling of ablation components, according to at least some aspects of the present disclosure.

FIGS. 14A, 14B, and 14C are perspective view examples of end-to-side coupling of ablation components respectively in first, second, and third positions, all according to at least some aspects of the present disclosure.

FIGS. 15A, 15B, and 15C are perspective view examples of end-to-side coupling of ablation components respectively in first, second, and third positions, all according to at least some aspects of the present disclosure.

FIG. 16 is a perspective view of example side-to-side coupling of ablation components, according to at least some aspects of the present disclosure.

FIG. 17 is a perspective view of example ablation components 1700, 1750, according to at least some aspects of the present disclosure.

FIGS. 18A and 18B are perspective view examples of ablation components, all according to at least some aspects of the present disclosure.

FIG. 19 is simplified schematic view of an example ablation system in use on a heart, according to at least some aspects of the present disclosure.

FIG. 20 is a simplified perspective view of example magnetic elements, according to at least some aspects of the present disclosure.

FIG. 21 is a simplified perspective view of an example magnetic element, according to at least some aspects of the present disclosure.

FIG. 22 is a simplified perspective view of an example magnetic element, according to at least some aspects of the present disclosure.

FIG. 23A is a simplified elevation view of an example magnetic element, according to at least some aspects of the present disclosure.

FIG. 23B is a simplified plan view of another example magnetic element, according to at least some aspects of the present disclosure.

FIG. 24 is a simplified cross-sectional view of example cooperating magnetic elements, according to at least some aspects of the present disclosure.

FIGS. 25A and 25B are simplified cross-sectional views of example cooperating magnetic elements, all according to at least some aspects of the present disclosure.

FIG. 26A is a simplified perspective view of an example magnetic element, according to at least some aspects of the present disclosure.

FIG. 26B is a simplified cross-sectional view, of example cooperating magnetic elements, according to at least some aspects of the present disclosure.

FIG. 27A is a simplified cross-sectional view of example cooperating magnetic elements, according to at least some aspects of the present disclosure.

FIG. 27B is a simplified cross-sectional view of an example electromagnet, according to at least some aspects of the present disclosure.

FIG. 27C is a simplified cross-sectional view of an alternative example electromagnet, according to at least some aspects of the present disclosure.

FIG. 28A is a simplified elevation view of an example expandable magnetic element in an expanded configuration, according to at least some aspects of the present disclosure.

FIG. 28B is a detailed elevation view of an example expandable portion of the magnetic element of FIG. 28A in a retracted configuration, according to at least some aspects of the present disclosure.

FIG. 28C is a detailed elevation view of the example expandable portion of the magnetic element of FIG. 28A in an expanded configuration, according to at least some aspects of the present disclosure.

FIG. 29 is a simplified schematic view of an example magnetic element, according to at least some aspects of the present disclosure.

FIG. 30 is a simplified cross-sectional view of example magnetic elements, according to at least some aspects of the present disclosure.

FIG. 31 is a simplified cross-sectional view of an example ablation system, according to at least some aspects of the present disclosure.

FIG. 32 is a simplified perspective section view of example cooperating magnetic elements, according to at least some aspects of the present disclosure.

FIG. 33 is a simplified perspective view of example cooperating magnetic elements, according to at least some aspects of the present disclosure.

### DETAILED DESCRIPTION OF THE INVENTION

The present disclosure contemplates that heart arrhythmias affect millions of people in the United States alone. For example, atrial fibrillation is a heart arrhythmia in which the electrical activity of the heart is disorganized. Common arrhythmias include ventricular tachycardia and/or inappropriate sinus tachycardia.

The present disclosure contemplates that effective treatments for cardiac arrhythmias may include creating a pattern (“maze”) of scar tissue (lesions) on a patient's heart to interrupt the errant electrical impulses in the heart tissue. The lesions may be created by cutting the cardiac tissue and sewing it back together. Alternatively, the lesions may be created by ablating the cardiac tissue using a variety of modalities, such as RF energy ablation, pulsed field ablation, cryoablation, ultrasound ablation, and/or laser ablation, for example. As used here, “ablation” may refer to the removal or destruction of a function of a body part, such as cardiac tissue, regardless of the apparatus or process used to carry out the ablation.

The present disclosure contemplates that, in some circumstances, it may be desirable for cardiac ablation lesions to span the entire thickness of the heart tissue (a heart transmural lesion). Thus, some ablation apparatus and methods may be configured to create one or more transmural lesions. As used herein, “transmural” may refer to through the wall or thickness of tissue, such as through the wall or thickness of a hollow organ or vessel.

The present disclosure contemplates that because RF ablation delivers energy between two electrodes to heat the target tissue, an effective electrode configuration for achieving transmural lesions with RF energy may include ablation electrodes positioned on one or multiple sides of the tissue targeted for ablation. Similar approaches may be utilized with other ablation modalities.

In the following detailed description of examples of the disclosure, specific examples in which the various aspects of the disclosure may be practiced are described in sufficient detail to enable those skilled in the art to practice the invention, and it is to be understood that other examples may be utilized and that logical, architectural, programmatic, mechanical, electrical, and other changes may be made without departing from the spirit or scope of the present disclosure. The following detailed description is, therefore, not to be taken in a limiting sense, and the scope of the present disclosure is defined by the appended claims and equivalents thereof. Within the descriptions of the different views of the figures, similar elements are provided similar names and reference numerals as those of the previous figure(s). The specific numerals assigned to the elements are provided solely to aid in the description and are not meant to imply any limitations (structural or functional or otherwise) on the described example. It will be appreciated that for simplicity and clarity of illustration, elements illustrated in the figures have not necessarily been drawn to scale. For example, the dimensions of some of the elements are exaggerated relative to other elements.

It is understood that the use of a specific component, device and/or parameter names, such as those of the executing utility, logic, and/or firmware described herein, are for illustrations only and not meant to imply any limitations on the described examples. The examples may thus be described with different nomenclature and/or terminology utilized to describe the components, devices, parameters, methods and/or functions herein, without limitation. References to any specific protocol or proprietary name in describing one or more elements, features or concepts of the examples are provided solely on an illustrative basis of one implementation, and such references do not limit the extension of the claimed limitations to examples in which a different element, feature, protocol, or concept name is utilized. Thus, each term utilized herein is to be given its broadest interpretation given the context in which that term is utilized.

As further described below, implementation of the functional features of the disclosure described herein is provided within processing devices and/or structures and can involve use of a combination of hardware, firmware, as well as several software-level constructs (e.g., program code and/or program instructions and/or pseudo-code) that execute to provide a specific utility for the device or a specific functional logic. The presented figures may illustrate both hardware components and software and/or logic components.

Those of ordinary skill in the art will appreciate that the hardware components and basic configurations depicted in the figures may vary. The illustrative components are not intended to be exhaustive, but rather are representative to highlight essential components that are utilized to implement aspects of the described examples. For example, other devices/components may be used in addition to or in place of the hardware and/or firmware depicted. The depicted examples are not meant to imply architectural or other limitations with respect to the presently described aspects and/or the general invention. The description of the illustrative examples can be read in conjunction with the accompanying figures. Examples incorporating teachings of the present disclosure are shown and described with respect to the figures presented herein.

Unless specifically indicated, it will be understood that the description of the structure, function, and/or methodology with respect to any illustrative example herein may apply to any other illustrative example. More generally, it is within the scope of the present disclosure to utilize any one or more features of any one or more examples described herein in connection with any other one or more features of any other one or more other example(s) described herein. Accordingly, any combination of any of the features or examples described herein is within the scope of this disclosure.

### Permanent Magnet Coupled Ablation Components

Examples according to the present disclosure may be described and illustrated to encompass devices, methods, and techniques relating to medical procedures. Of course, it will be apparent to those of ordinary skill in the art that the examples discussed below are exemplary and may be reconfigured by removing and/or adding elements of other examples without departing from the scope and spirit of the present disclosure. It is also to be understood that variations of the examples contemplated by one of ordinary skill in the art shall concurrently comprise part of the instant disclosure. However, for clarity and precision, the examples as discussed below may include optional steps, methods, and/or features that one of ordinary skill should recognize as not being a requisite to fall within the scope of the present disclosure.

The present disclosure encompasses, among other things, medical instruments and devices and related methods, and, more specifically, devices for ablating tissue and related methods. Some examples according to at least some aspects of the present disclosure may be useful in connection with ablation of cardiac tissue, such as to treat cardiac arrhythmias like atrial fibrillation, ventricular tachycardia, and/or inappropriate sinus tachycardia. Some examples according to at least some aspects of the present disclosure may be configured to deliver ablation energy, such as through a heart wall to create an efficient lesion (that may be transmural), while minimizing collateral tissue trauma.

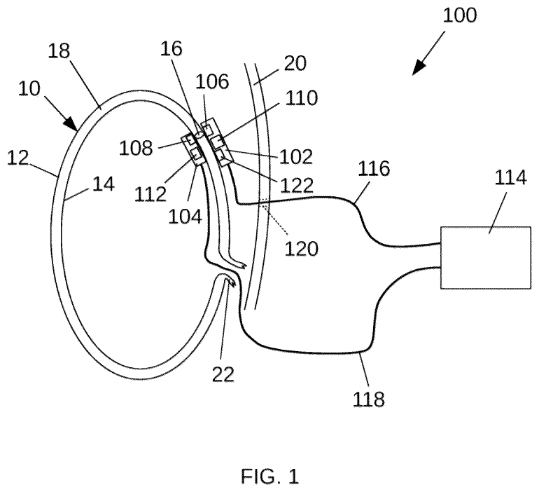

FIG. 1 is a simplified schematic view of an example ablation system 100 in use on a heart 10, according to at least some aspects of the present disclosure. The example ablation system 100 may include a first ablation component 102 configured to be positioned on a first side of a target tissue and/or a second ablation component 104 configured to be positioned on a second side of the target tissue. The first and second sides of the target tissue may be opposite sides.

For example, the first ablation component 102 may be configured to be positioned on an epicardial (exterior) surface 12 of the heart 10. The second ablation component 104 may be configured to be positioned on an endocardial (interior) surface 14 of the heart 10.

The first ablation component 102 may include at least one ablation element 106, such as an electrode, and the second ablation component 104 may include at least one ablation element 108, such as an electrode. The ablation elements 106, 108 may be configured to cooperate to create a lesion 16 in the target tissue, such as a transmural lesion extending through the full thickness of the heart wall 18.

The first ablation component 102 may include at least one magnetic element 110 and the second ablation component 104 may include at least one magnetic element 112. The magnetic elements 110, 112 may be configured to cooperate to facilitate positioning the first ablation component 102 and/or the second ablation component 104. For example, the magnetic elements 110, 112 may be utilized to align the ablation elements 106, 108 across the heart wall 18 to create the lesion 16 at the desired location. Some examples may utilize selective coupling and/or uncoupling of the magnetic elements 110, 112 to facilitate positioning the electrodes as well as for subsequent repositioning to nearby target tissues for subsequent ablations. Some example ablation components 102, 104 may be configured to at least partially compress tissue therebetween. For example, the magnetic elements 110, 112 may be configured to cooperate to compress the target tissue (e.g., heart wall 18) between the ablation components 102, 104, which may improve ablation outcomes in some circumstances.

The example ablation system 100 may include a control unit 114. The control unit 114 may be operable to control the operation of the ablation components 102, 104. In some examples, the control unit 114 may be configured to selectively provide ablation energy (e.g., RF energy from an electrosurgical generator) to electrodes comprising the ablation elements 106, 108. In some examples, the control unit 114 may be configured to control the operation of the magnetic elements 110, 112. For example, the control unit 114 may be configured to selectively apply electrical current through one or more electromagnets comprising one or more of the magnetic elements 110, 112. The ablation components 102, 104 may include respective connecting elements 116, 118, which may operatively couple aspects of the ablation components 102, 104 such as, without limitation, the ablation elements 106, 108 and/or the magnetic elements 110,112, to the control unit 114. The connecting elements 116, 118 may include or be used in connection with one or more electrical conductors, fluidic conduits, mechanical force transmission components, catheters, wires, steerable portions, sheaths, trocars, hemostatic components, etc.

In some examples, the exemplary ablation system 100 may be configured to access the target tissue (e.g., heart wall 18) via a surgical approach (open-chest and/or minimally invasive) and/or via the patient's vascular system. For example, the first ablation component 102 may be positioned on the epicardial surface 12 via a percutaneous or surgical sub-xiphoid or intercostal access or incision 120, such as through the chest wall 20. The second ablation component 104 may be positioned on the endocardial surface 14 via a blood vessel 22 (e.g., the femoral vein). In various circumstances, the routes used to obtain access to the epicardial and endocardial surfaces may be determined based at least in part upon factors such as the portion of the heart wall 18 that comprises the target tissue for the ablation, the patient's potential anatomical variations, previous surgeries, etc. In some examples, either or both of the first ablation component 102 and the second ablation component 104 may access the target tissue by any suitable route, such as open chest, thoracotomy, sub-xiphoid, trans-diaphragmatic, and/or vascular access.

In some examples, one or more of the ablation components 102, 104 may include and/or may be configured to operate in connection with a device configured to selectively and/or releasably attach to the target tissue, such as a tissue anchor. For example, the first ablation component 102 may include a vacuum anchor 122, which may be configured to selectively and/or releasably secure the first ablation component 102 to the epicardial surface 12. In some such examples, the connecting element 116 may include a vacuum line and/or the control unit 114 may include a vacuum pump and/or related control apparatus.

Generally, some examples may be operated as follows. Appropriate access to the epicardial 12 and endocardial 14 surfaces may be obtained. The first ablation component 102 may positioned at a desired location on the epicardial surface 12. The vacuum anchor 122 may be operated (e.g., vacuum may be applied) to secure the first ablation component 102 in position on the epicardial surface 12. The magnetic elements 110, 112 may be utilized (e.g., magnetically coupled) to position and/or secure the second ablation component 104 on the endocardial surface 14. For example, the magnetic element 110 of the first ablation component 102 may magnetically attract the magnetic element 112 of the second ablation component 104. The ablation elements 106, 108 may be operated to ablate the target tissue and create the lesion 16. The magnetic elements 110, 112 may be uncoupled. In some examples, the magnetic elements 110, 112 may be reconfigured into a magnetically repulsive arrangement, such as to disengage one or more of the first ablation component 102 and/or the second ablation component 104 from the target tissue and from one another. The vacuum anchor 122 may be released. The first and second ablation components 102, 104 may be withdrawn from the patient.

In some examples, an ablation component may be passively oriented and/or located on the target tissue. For example, the ablation component may be generally positioned on the target tissue, such as by direct imaging and/or through a medical scope. The ablation component may be releasably attached to the target tissue, such as by a vacuum anchor or other tissue anchor. Related elements associated with the ablation component may include a single lumen sheath, a multilumen sheath, and/or an inflatable element, such as a space-making element. In some examples, an ablation component may be oriented and/or located on the target tissue using an image guided catheter. For example, the ablation component and/or the cooperating ablation component may comprise guidance features. In some examples, an ablation component may include sensing features and/or stimulation features and/or may be used in connection with a cooperating ablation component including sensing features and/or stimulation features.

Some examples may provide sensing capabilities for various parameters. For example, tissue impedance may be sensed between ablation electrodes, such as to assess the extent of an ablation. In some examples, measurements of magnetic fields may be utilized, such as to determine a distance between certain components. In some example, one or more temperatures may be measured.

The foregoing description of the example ablation system 100 provides context for various examples described below. The various examples described below may comprise components of the example ablation system 100, or may be utilized in alternative systems. Any feature or component of any example described herein may be utilized in connection with any other example, and repeated description of components is omitted for brevity.

The ablation elements 106, 108 and magnetic elements 110, 112 are illustrated as separate elements in FIG. 1 for clarity. However, in some examples, one or more of the ablation elements 106, 108 and/or one or more of the magnetic elements 110, 112 may be combined into a single element. For example, at least a portion of a magnetic element 110, 112 may also act as an ablation element 106, 108 (e.g., an RF electrode), or a dedicated electrode material may be used around or adjacent to a magnetic element 110, 112.

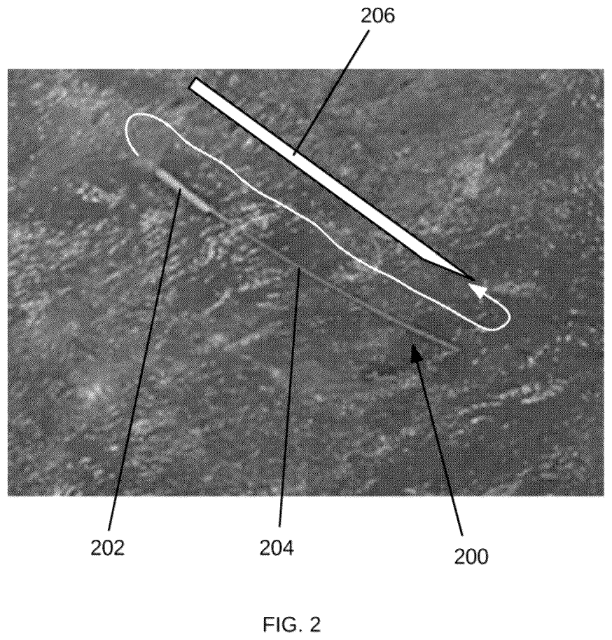

FIG. 2 is a perspective view of an example ablation component 200, according to at least some aspects of the present disclosure. The ablation component 200 may comprise a combined ablation/magnetic element 202. For example, the ablation/magnetic element 202 may be formed of a ferrous material, which may be electrically conductive to deliver RF energy for ablation and/or which may be ferromagnetic. The ablation/magnetic element 202 may be disposed on an elongated wire 204.

In some examples, the ablation component 200 may be positioned on the endocardial surface 14 to act as a second ablation component 104 as described above with reference to FIG. 1. In some such examples, the ablation/magnetic element 202 may be magnetically attracted by the magnetic element 110 of the first ablation component 102, which may be positioned on the epicardial surface 12. The ablation/magnetic element 202 may cooperate with the ablation element 106 of the first ablation component 102 to deliver RF energy to ablate the heart wall generally interposing the ablation element 106 of the first ablation component 102 and the ablation/magnetic element 202 of the ablation component 200.

In some examples, the ablation component 200 may be introduced via the pericardial space. The ablation component 200 may be delivered through the heart wall 18 via a needle or through a hollow needle 206, such as a small bore (e.g., 21 gauge) needle. In some examples, the hole created by delivering the ablation component 200 in this manner may be small enough not to require hemostasis during ablation and/or suture upon removal.

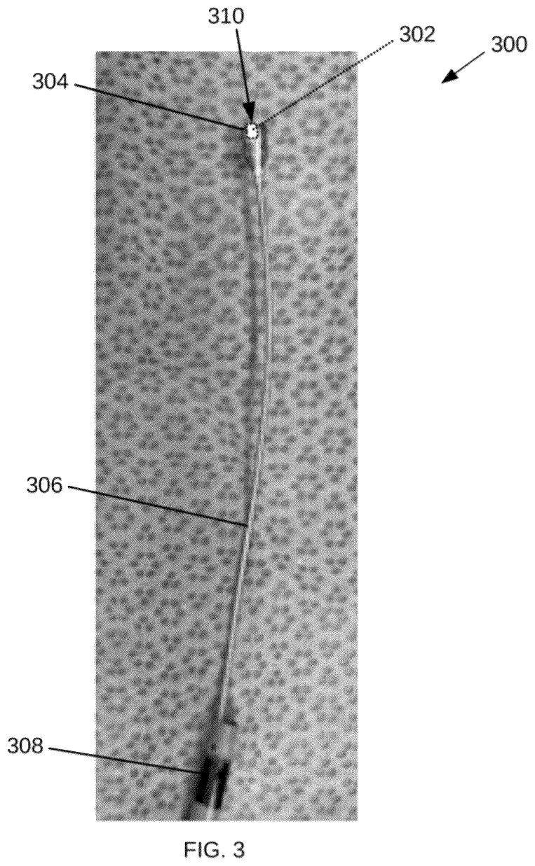

FIG. 3 is a perspective view of an example ablation component 300, according to at least some aspects of the present disclosure. Ablation component 300 may comprise one or more magnetic elements 302 (e.g., a permanent magnet, electromagnetic, etc.) disposed generally within one or more ablation elements 304 (e.g., an electrode). The magnetic element 302 may be positioned generally proximate the distal end of the ablation element 304 and/or its magnetic field may be aligned generally longitudinally. As used herein, “distal” may refer to a direction generally away from an operator of a system or device (e.g., a cardiac surgeon, an interventional cardiologist, a robot, etc.), such as toward the distant-most end of a device that is inserted into a patient's body. The magnetic element 302 and the ablation element 304 may be disposed proximate a distal end of a connecting element, such as a steerable catheter 306, which may extend proximally and/or may operably connect to a control unit 114 (see FIG. 1). As used herein, “proximal” may refer to a direction generally toward an operator of a system or device, such as away from the distant-most end of a device that is inserted into a patient's body. In some examples, the catheter 306 may have sufficient rigidity and/or steerability to allow positioning of the magnetic element 302 and ablation element 304 without substantial external support. The ablation component 300 may be routed through a larger diameter catheter 308, such as a steerable guide sheath. For example, the catheter 308 may be used to deliver the ablation component 300 to the endocardial surface 14 of a patient's heart 10, such as via the patient's vascular system. In some examples, the ablation component 300 may comprise the second ablation component 104 described above with reference to FIG. 1. In some examples, the ablation component 300 may be configured for use with a distal end face 310, which may be substantially flat, directed to the target tissue.

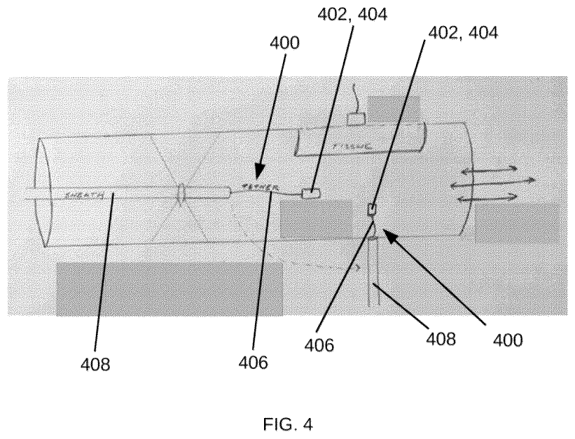

FIG. 4 is an elevation view of an example ablation component 400 in two orientations, according to at least some aspects of the present disclosure. Ablation component 400 may be generally similar to ablation component 300 described above and repeated description is omitted for brevity. Ablation component 400 may comprise one or more magnetic elements 402 and/or one or more ablation elements 404 disposed distally on a flexible tether 406 instead of the steerable catheter 306 of ablation component 300 described above. In some examples, the tether 406 may be sufficiently flexible (e.g., “floppy”) so that it is generally incapable of maintaining the position of the magnetic element 402 and ablation element 404 without external support. The ablation component 400 may be routed through a catheter 408, such as a steerable guide sheath. In some examples, the catheter 408 may comprise a multi-lumen catheter that allows an epicardial element to readily move from location to location. For example, the catheter 408 may be used to deliver the ablation component 400 to the endocardial surface 14 of a patient's heart 10, such as via the patient's vascular system. In some examples, the ablation component 400 may comprise the second ablation component 104 described above with reference to FIG. 1.

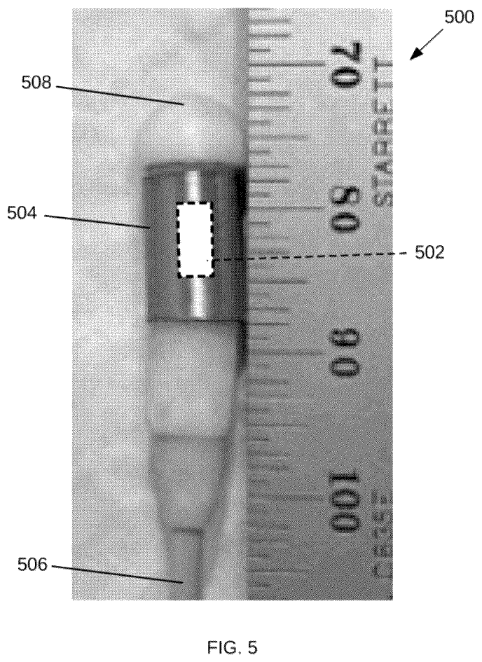

FIG. 5 is a perspective view of an example ablation component 500, according to at least some aspects of the present disclosure. Ablation component 500 may comprise one or more magnetic elements 502 (e.g., a permanent magnet, electromagnetic, etc.) disposed at least partially within one or more ablation elements 504 (e.g., a generally cylindrical ring electrode). The ablation elements may be electrically shielded on one or more of the sides that will not contact tissue to prevent shunting of electrical energy through blood, and the magnetic element may only exist on the one or more sides designed to contact tissue. The ablation element may also have a window or cutout through which the magnet within can connect with the opposing magnet without the electrode diminishing the magnetic field. The magnetic element 502 may be positioned generally radially within the ablation element 504, may be positioned generally between the longitudinal distal and proximal ends of the ablation element 504, and/or may be generally diametrically magnetized. The magnetic element 502 may be configured to cause a lateral/radial force to be applied directly at the electrode, which may be generally toward where an ablation is performed. In some alternative examples, the magnetic element 502 may be configured to cause a lateral/radial force to be applied directly at the electrode, which may be generally away from where an ablation is performed. The magnetic element 502 and the ablation element 504 may be disposed proximate a distal end of a connecting element 506, such as one or more steerable catheters and/or wires, which may operably connect to a control unit 114 (see FIG. 1). The ablation component 500 may include a distal tip 508, which may be generally rounded or other atraumatic shape, distal to the ablation element 504. The ablation component 500 may be routed through a larger diameter catheter, such as a steerable guide sheath. The ablation component 500 may be positioned on the endocardial surface 14 of a patient's heart 10, such as via the patient's vascular system. In some examples, the ablation component 500 may comprise the second ablation component 104 described above with reference to FIG. 1.

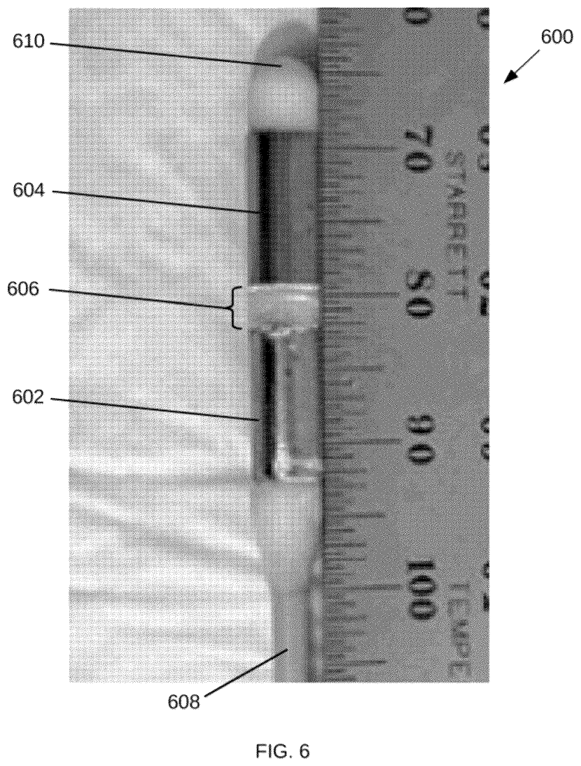

FIG. 6 is a perspective view of an example ablation component 600, according to at least some aspects of the present disclosure. Ablation component 600 may comprise one or more magnetic elements 602 (e.g., a permanent magnet, electromagnetic, etc.) disposed generally longitudinally relative to one or more ablation elements 604. For example, the magnetic element 602 may comprise a generally cylindrical permanent magnet that is positioned proximally with respect to an ablation element 604 comprising a generally cylindrical ring electrode. In alternative examples, the magnetic element 602 may be positioned distally relative to the ablation element 604. The magnetic element 602 and the ablation element 604 may be positioned substantially adjacent (e.g., minimal or no gap therebetween), or may be spaced apart by a longitudinal distance 606. The magnetic element 602 may be generally diametrically magnetized. The magnetic element 602 and the ablation element 604 may be disposed proximate a distal end of a connecting element 608, such as one or more steerable catheters and/or wires, which may operably connect to a control unit 114 (see FIG. 1). The ablation component 600 may include a distal tip 610, which may be generally rounded or embody another atraumatic shape, distal to the ablation element 604. The ablation component 600 may be routed through a larger diameter catheter, such as a steerable guide sheath. The ablation component 600 may be positioned on the endocardial surface 14 of a patient's heart 10, such as via the patient's vascular system. In some examples, the ablation component 600 may comprise the second ablation component 104 described above with reference to FIG. 1. In other examples, the ablation component 600 may comprise the first ablation component 102 described above with reference to FIG. 1 and/or may be used epicardially.

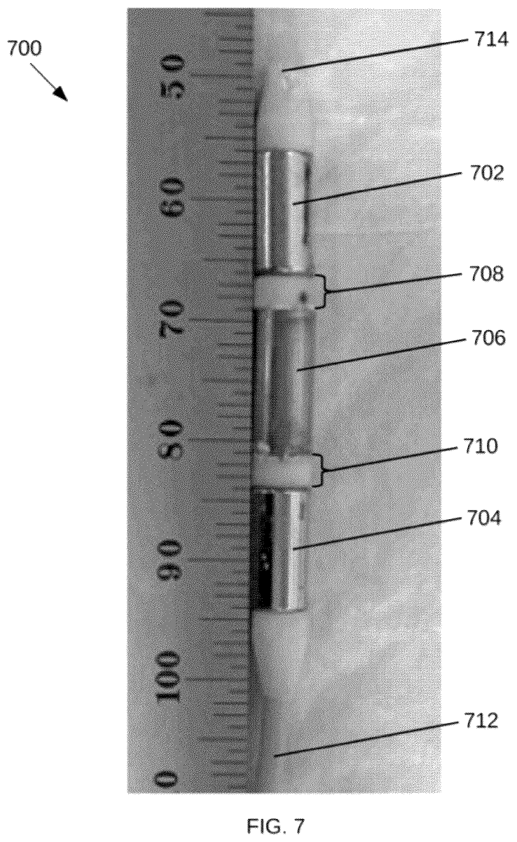

FIG. 7 is a perspective view of an example ablation component 700, according to at least some aspects of the present disclosure. Ablation component 700 may comprise two or more magnetic elements 702, 704 disposed generally longitudinally relative to one or more ablation elements 706. For example, the magnetic elements 702, 704 may each comprise generally cylindrical permanent magnets. One magnetic element 702 may be positioned distally relative to the ablation element 706 and/or the other magnetic element 704 may be positioned proximally relative to the ablation element 706.

The ablation element 706 may comprise a generally cylindrical ring electrode. In alternative examples, two or more magnetic elements 702, 704 may be positioned distally or proximally relative to the ablation element 706. The magnetic elements 702, 704 and the ablation element 706 may be positioned substantially adjacent (e.g., minimal or no gaps therebetween), or may be spaced apart by longitudinal distances 708, 710. The magnetic elements 702, 704 may be generally diametrically magnetized. In some examples, there is a sequence of magnet-electrode-magnet-electrode-magnet etc., and/or may be incorporated on a straight axis, as shown in FIG. 7, or may be configured on a catheter with an intrinsic bias to a curved shape, like a circle or half circle. The magnetic elements 702, 704 and the ablation element(s) 706 may be disposed proximate a distal end of a connecting element 712, such as one or more steerable catheters and/or wires, which may operably connect to a control unit 114 (see FIG. 1). The ablation component 700 may include a distal tip 714, which may be generally rounded or embody another atraumatic shape, distal to the magnetic element 702. The ablation component 700 may be routed through a larger diameter catheter, such as a steerable guide sheath. The ablation component 700 may be positioned on the endocardial surface 14 of a patient's heart 10, such as via the patient's vascular system. In some examples, the ablation component 700 may comprise the second ablation component 104 described above with reference to FIG. 1. In other examples, the ablation component 700 may comprise the first ablation component 102 described above with reference to FIG. 1 and/or may be used epicardially.

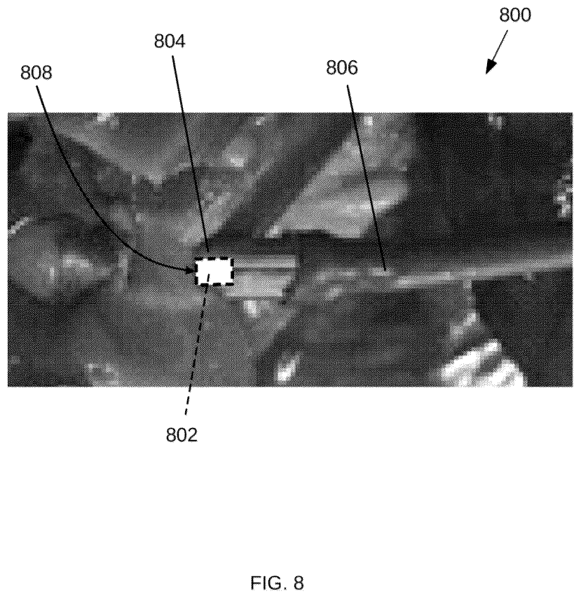

FIG. 8 is a perspective view of an example ablation component 800, according to at least some aspects of the present disclosure. Ablation component 800 may be generally similar to ablation component 300 described above and/or may comprise one or more magnetic elements 802 (e.g., permanent magnet(s) and/or electromagnet(s)) disposed generally within one or more ablation elements 804 (e.g., an electrode). The magnetic element 802 may be positioned generally proximate the distal end of the ablation element 804 and/or its magnetic field may be aligned generally longitudinally/axially.

The magnetic element 802 and the ablation element 804 may be disposed proximate a distal end of a shaft 806. In some examples, the shaft 806 may be substantially straight. In some examples, the shaft 806 may be curved and/or may be flexible and/or malleable. In some examples, the shaft 806 may be articulable, such as to allow repositioning (e.g., steering) of the magnetic element 802 and/or the ablation element 804 relative to the target tissue.

The ablation component 800 may be positioned on the epicardial surface 12 of a patient's heart 10, such as via a sub-xiphoid or intercostal incision or access device. In some examples, the ablation component 800 may comprise the first ablation component 102 described above with reference to FIG. 1. In some examples, the ablation component 800 may be configured for use with a distal end face 808 directed to the target tissue.

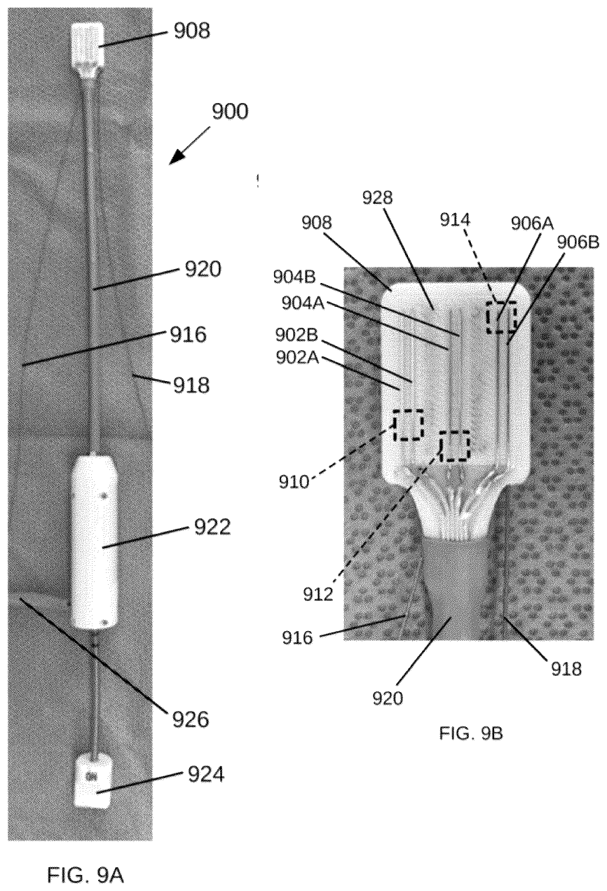

FIG. 9A is a perspective view of an example ablation component 900 and FIG. 9B is a detailed perspective view of a distal portion of the ablation component 900, all according to at least some aspects of the present disclosure. Ablation component 900 may include one or more ablation elements, such as elongated electrodes (e.g., copper rail electrodes) 902A, 902B, 904A, 904B, 906A, 906B, which may be disposed substantially in a generally parallel, generally longitudinal orientation on a substantially rigid, generally flattened pad 908. Although FIGS. 9A and 9B illustrate an example comprising three pairs of electrodes, it will be understood that alternative examples may include one or more ablation elements, such as one or more individual electrodes or pairs of electrodes. In some examples, respective pairs of electrodes may be configured to act as a single electrode, such as with the Isolator Linear Pen or the Coolrail Linear Pen (available from AtriCure, www.atricure.com).

Ablation component 900 may include one or more magnetic elements 910, 912, 914, which may each be associated with a respective pair of electrodes 902A, 902B, 904A, 904B, 906A, 906B and/or which may be disposed within the pad 908. In some examples, an individual magnetic element 910, 912, 914 may be inserted into a port extending within the pad 908 and associated with one of the pairs of electrodes 902A, 902B, 904A, 904B, 906A, 906B. In some examples, the magnetic elements 910, 912, 914 may be movable (e.g., longitudinally) along at least a portion of the length of the electrodes 902A, 902B, 904A, 904B, 906A, 906B. In FIG. 9B, the magnetic elements 910, 912, 914 are shown in different longitudinal positions. Markings, such as visual indicia on a handle or actuator, or on a visual display, may be used to indicate the longitudinal position of one or more of the magnetic elements 910, 912, 914 to the user. Some such magnetic elements 910, 912, 914 may be disposed on stiff shafts 916, 918 to facilitate such operations. In some examples, the stiff shafts 916, 918 may act as a guide over which the magnetic elements 910, 912, 914 may be steered into place. Alternatively, the magnetic elements 910, 912, 914 may move down a central lumen and be steered into location under a preferred electrode. The preferred track may also be selectable from the handle via a divertor/director, which may be operable from the handle. In some examples, the magnetic elements 910, 912, 914 may be rotatable about a longitudinal/axial axis of the pad to reorient the magnetic poles of the magnetic element 910, 912, 914. Such examples may facilitate selective attraction and repulsion of a cooperating magnet in another ablation component.

For example, ablation component 900 may be positioned on the epicardial surface 12 of a patient's heart 10, such as via a sub-xiphoid or intercostal incision or access device and/or may comprise the first ablation component 102 described above with reference to FIG. 1. One or more of the magnetic elements 910, 912, 914 of the ablation component 900 may be rotated to selectively attract or repel the magnetic element 112 of the second ablation component 104 of FIG. 1. In some examples, the ablation component 900 may be configured for use with a lateral side face 928 directed to the target tissue.

The pad 908 may be disposed proximate a distal end of a shaft 920. In some examples, the shaft 920 may be substantially straight. In some examples, the shaft 920 may be curved and/or may be flexible and/or bendable. In some examples, the shaft 920 may be articulable, such as to allow repositioning of the pad 908 relative to the target tissue. The ablation component 900 may include a handle 922 positioned generally proximally on the shaft 920. The handle 922 may include and/or may be operatively coupled to one or more actuators 924, which may be configured to control various aspects of the operation of the ablation component 900, such as steering. The ablation component 900 may include a connecting component 926, which may be operatively coupled to the control unit 114. In some examples, the ablation component 900 may comprise the second ablation component 104 described above with reference to FIG. 1 and/or may be used endocardially.

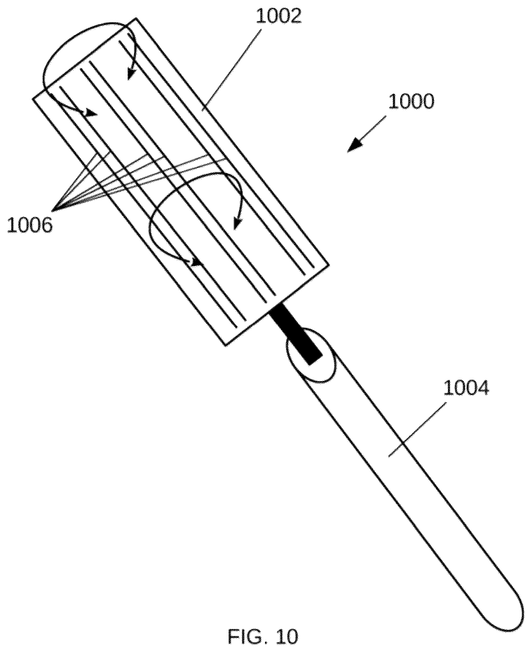

FIG. 10 is a simplified perspective view of an ablation component 1000, according to at least some aspects of the present disclosure. Ablation component 1000 may be generally similar to ablation component 900, described above, except that it may comprise an at least partially flexible pad 1002. For example, the flexible pad 1002 may comprise a flexible backer. Ablation component 1000 may include ablation elements 1006, magnetic elements, and other features similar to those described herein for other examples and repeated description is omitted for brevity.

In some examples, the pad 1002 may be configured to curl about a generally longitudinal/axial axis so that it can be delivered and recovered through a catheter 1004. In some examples, the deployed (uncurled, unfurled, and/or unfolded) lateral width of the pad 1002 may be substantially greater than the internal diameter of the catheter 1004. Some example ablation devices 1000 may be positioned on the epicardial surface 12 of a patient's heart 10, such as via a sub-xiphoid or intercostal incision or access device and/or may comprise the first ablation component 102 described above with reference to FIG. 1. For example, the catheter may be routed to the epicardial surface 12 and the ablation device 1000 may be deployed from the catheter. After the ablation operations are complete, the ablation device 1000 may be curled, furled, and/or folded and withdrawn into or through the catheter for removal from the patient's body.

FIG. 11A is a detailed perspective view of a portion of an ablation component 1100 and FIG. 11B is a perspective view of an ablation component 1150, all according to at least some aspects of the present disclosure. Ablation components 1100, 1150 may be generally similar to ablation component 1000, described above, except that they may comprise one or more inflatable elements (e.g., balloons) 1102, 1152 instead of the flexible pad 1002. Ablation components 1100, 1150 may include ablation elements 1104, 1156, magnetic elements 1106, 1158, and other features similar to those described herein for other examples and repeated description is omitted for brevity. In some examples, ablation components, such as electrodes, may be printed and/or embedded on the inflatable elements 1102, 1152. Inflating the inflatable elements 1102, 1152 may act to increase spacing between the ablation elements 1104, 1156, for example. In some examples, such as shown in FIG. 11A, the ablation elements 1104 and the magnetic elements 1106 may be arranged in a generally alternating matrix arrangement. For example, such a configuration may be used with an ablation component similar to ablation component 700 described above with reference to FIG. 7. In some examples, such as shown in FIG. 11B, the ablation elements 1156 may comprise generally elongated electrodes, the magnetic elements 1158 may be generally elongated, and/or ablation elements 1156 and the magnetic elements 1158 may be arranged in an alternating arrangement. Some such examples may be utilized similarly to those described and depicted in FIGS. 9A and 9B. Some examples may comprise combined ablation/magnetic elements.

In some examples, the inflatable elements 1102, 1152 may be configured to move between a deflated configuration and an inflated configuration. For example, a fluid (e.g., gas or liquid such as saline solution) may be directed into and/or withdrawn from the interior of the inflatable elements 1102, 1152 from a pressure source (e.g., syringe 1154). In some examples, the inflated size of the inflatable element 1102, 1152 may be substantially larger than the internal diameter of a device used to direct the inflatable element 1102, 1152 to the ablation site (e.g., a catheter). Some example ablation devices 1100, 1150 may be positioned on the epicardial surface 12 of a patient's heart 10, such as via a sub-xiphoid or intercostal incision or access device and/or may comprise the first ablation component 102 described above with reference to FIG. 1. For example, a catheter may be routed to the epicardial surface 12 and the ablation device 1100, 1150 may be deployed from the catheter and/or inflated. After the ablation operations are complete, the ablation device 1100, 1150 may be deflated and/or withdrawn into or through the catheter for removal from the patient's body.

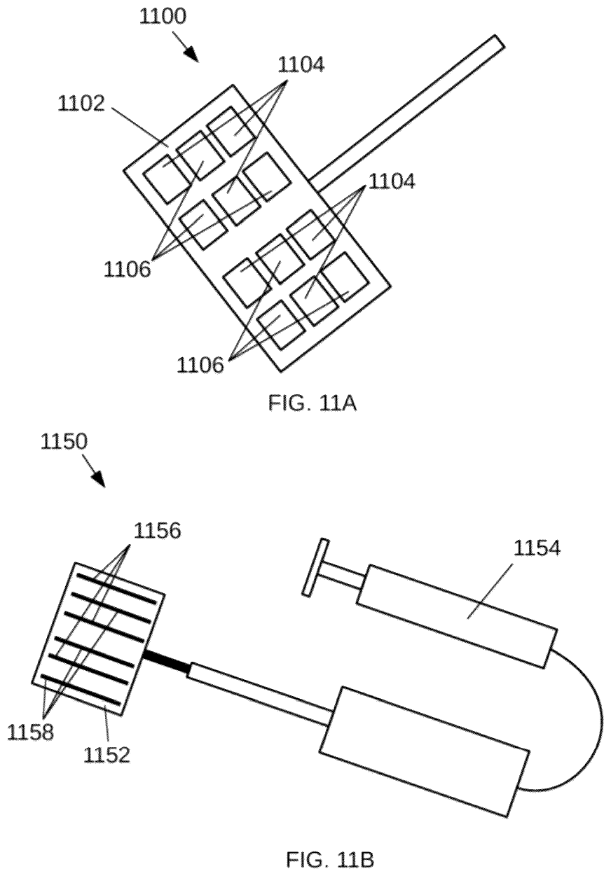

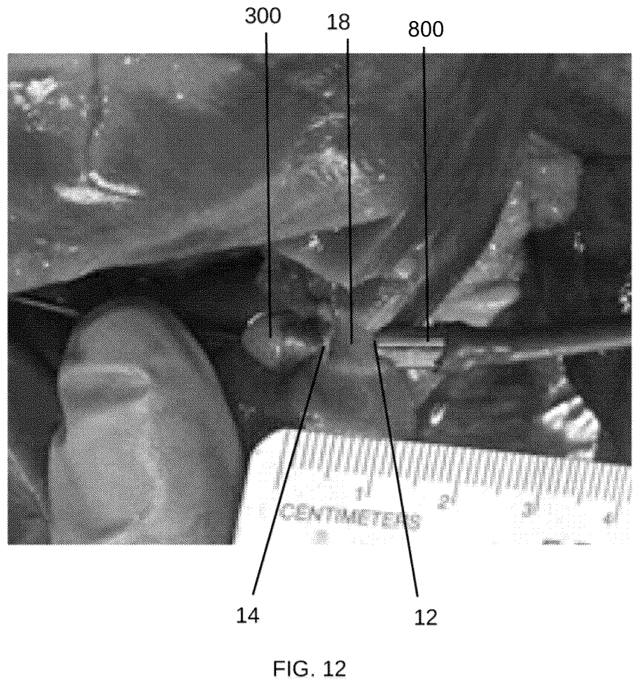

FIG. 12 is a perspective view of an example of end-to-end coupling of ablation components 300, 800, according to at least some aspects of the present disclosure. In this illustrative example, the ablation component 800 may comprise the first ablation component 102 described with reference to FIG. 1 and/or the ablation component 300 may comprise the second ablation component 104 described with reference to FIG. 1. The ablation components 300, 800 may be positioned in a distal end-to-distal end orientation across the heart wall 18. For example, the distal end face 310, 808 (see FIGS. 3, 8) of each ablation component 300, 800 may contact a respective surface 14, 12 of the heart wall 18 and/or the distal end faces of each ablation component 300, 800 may substantially face each other. The magnetic elements 302, 802 (see FIGS. 3, 8) may cooperate to align the ablation components 300, 800 and/or to compress the target tissue of the heart wall 18 between the ablation components 300, 800. In some examples, a distal end-to-distal end orientation may be used to create a spot lesion.

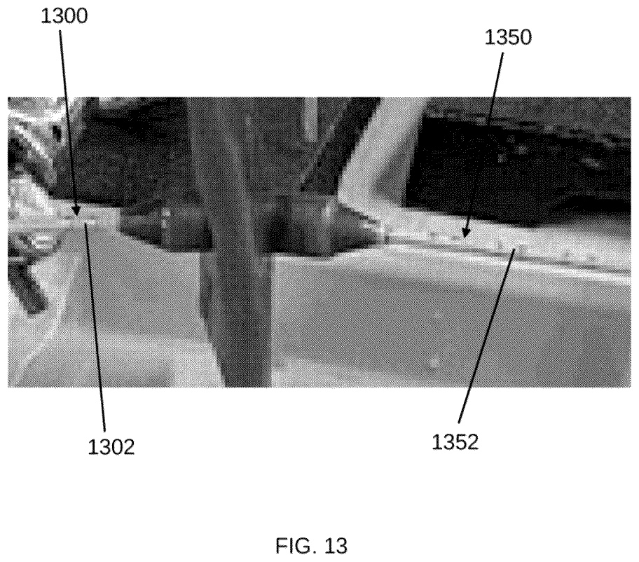

FIG. 13 is a perspective view of an example of distal end-to-distal end coupling of ablation components 1300, 1350, according to at least some aspects of the present disclosure. Ablation components 1300, 1350 may be generally similar to ablation components 300, 800, respectively, and may be utilized in a manner generally similar to that described above with reference to FIG. 12. Repeated description is omitted for brevity. In this example, each of the ablation components 1300, 1350 may include a shaft 1302, 1352, which may be steerable.

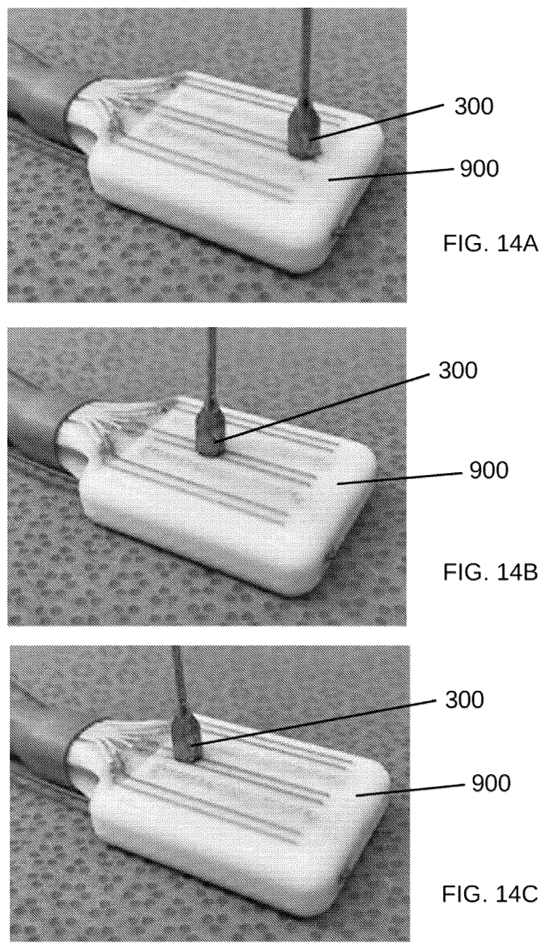

FIGS. 14A, 14B, and 14C are perspective views of exemplary distal end-to-distal end coupling of ablation components 300, 900, all according to at least some aspects of the present disclosure. For clarity, the target tissue (e.g., heart wall 18) interposing the ablation components 300, 900 is omitted in order to better show the positional relationship of the components in a coupled position, where the tissue would otherwise interpose the components. In this illustrative example, the ablation component 900 may comprise the first ablation component 102 described with reference to FIG. 1 and/or the ablation component 300 may comprise the second ablation component 104 described with reference to FIG. 1. The ablation components 300, 900 may be positioned in a distal end-to-side orientation across the heart wall 18. For example, the distal end face 310 of the ablation component 300 may contact the endocardial surface 14 of the heart wall 18 and/or the lateral side face 928 of the pad 908 may contact the epicardial surface 12 of the heart wall 18. The distal end face 310 of the ablation component 300 may face the lateral side face 928 of the pad 908 of the ablation component 900. The magnetic elements 302, 910, 912, 914 may cooperate to align the ablation components 300, 900 and/or to compress the target tissue (such as the heart wall 18) between the ablation components 300, 900. In some examples, a distal end-to-side orientation may be used to create a linear lesion. For example, magnetic element 302 may be moved along at least a portion of the length of the ablation elements 904A, 904B. As a result, the distal end face 310 of the ablation component 300 may move along the ablation elements 904A, 904B. The respective ablation elements may be continuously energized and/or may be energized and deenergized with the ablation component 300 in various positions as it is moved along the ablation elements 904A, 904B to create a generally linear lesion. A lesion, such as a linear lesion, may be formed by a single ablation operation and/or by a series of proximate (e.g., continuous and/or generally overlapping and/or adjacent) ablations that, together, form a lesion. As used herein, “a lesion” may comprise a plurality of substantially connected lesions. As used herein, “linear” may refer to an ablation lesion that is generally elongated and/or line-like, regardless of whether the lesion includes one or more generally straight portions and/or one or more curved or angled portions. For example, the structures illustrated in FIGS. 14A-14C may be configured to create one or more generally straight linear lesions.

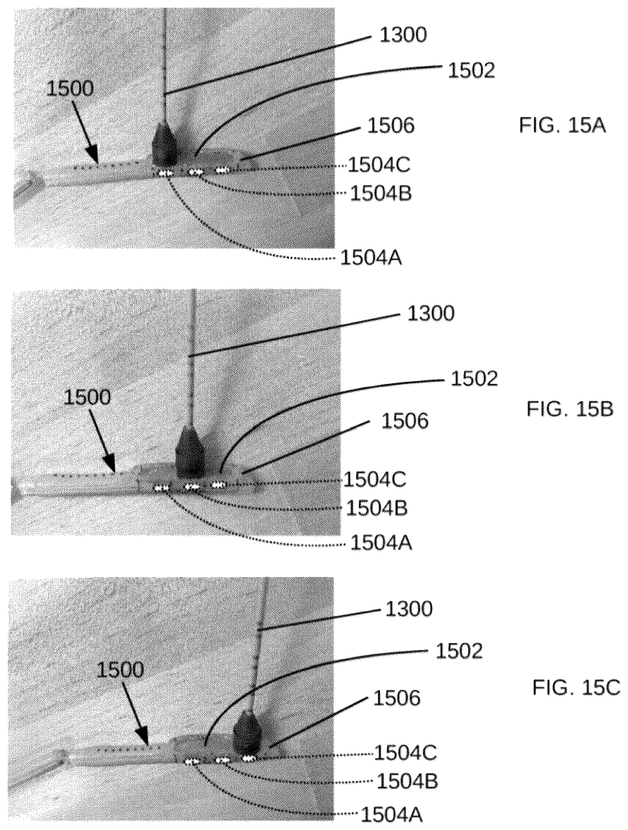

FIGS. 15A, 15B, and 15C are perspective view examples of distal end-to-side coupling of ablation components 1300, 1500, all according to at least some aspects of the present disclosure. Ablation component 1500 may be generally similar to ablation component 900 and may be utilized in a manner generally similar to that described above with reference to FIGS. 14A-14C. Repeated description is omitted for brevity. In this example, ablation component 1500 may include an ablation element comprising a plate electrode 1502 instead of the rail electrodes 902A, 902B, 904A, 904B, 906A, 906B of ablation component 900. In this example, the plate electrode 1502 may comprise a generally planar surface, which may be elongated in a generally distal-proximal longitudinal direction. Ablation component 1500 may include one or more magnetic elements 1504A, 1504B, 1504C positioned proximate the plate electrode 1502, such as inside a pad 1506, instead of the longitudinally movable magnetic elements 910, 912, 914 of ablation component 900. Although three magnetic elements 1504a, 1504b, 1504c are illustrated in FIGS. 15A-15C, it is within the scope of the disclosure to utilize any number of magnetic elements. Generally, ablation component 1300 may be moved along the plate electrode 1502 of the ablation component 1500. In some examples, the magnetic elements 1504a, 1504b, 1504c may comprise electromagnets which may be selectively energized to cooperate with the magnetic element(s) of the ablation component 1300 to facilitate movement and/or alignment of the ablation component 1300 relative to the ablation component 1500 to create a desired ablation lesion or series of lesions.

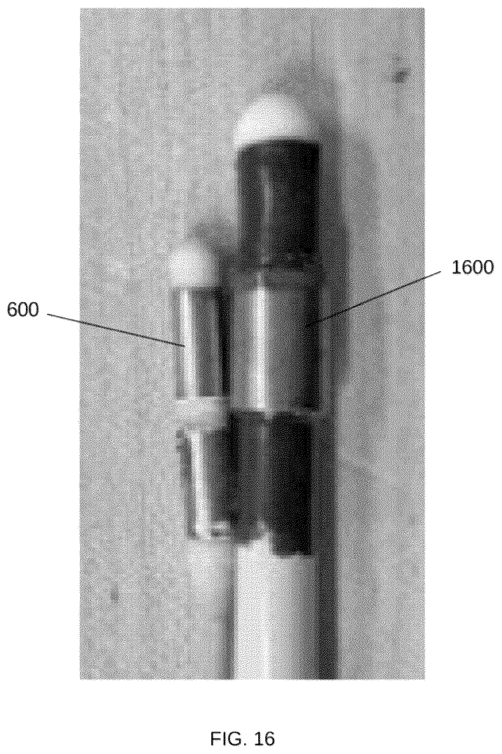

FIG. 16 is a perspective view of example side-to-side coupling of ablation components 600, 1600, according to at least some aspects of the present disclosure. For clarity, the target tissue (e.g., heart wall 18) interposing the ablation components 600, 1600 is omitted solely to better depict alignment of the depicted elements. In this illustrative example, ablation component 600 is positioned with its lateral side facing a corresponding lateral side of ablation component 1600. The ablation component 1600 may be generally similar to ablation components 500, 600, 700 and repeated description is omitted for brevity.

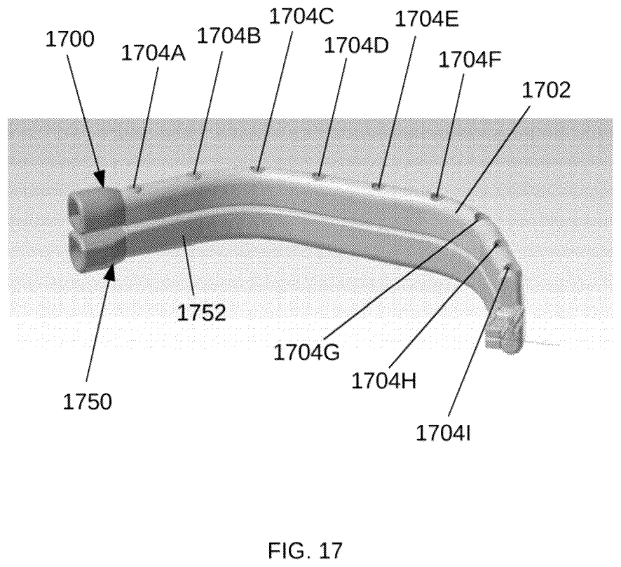

FIG. 17 is a perspective view of example ablation components 1700, 1750, according to at least some aspects of the present disclosure. In this example, each ablation component 1700, 1750 comprises a substantially rigid jaw 1702, 1752, which may be generally C-shaped. The target tissue (not shown in FIG. 17 for clarity) may be positioned between the jaws 1702, 1752. Ablation component 1700 may comprise magnetic elements 1704A-1704I, which may be disposed at least partially within the jaw 1702. Ablation component 1750 may include corresponding magnetic elements in a similar arrangement. The ablation components 1700, 1750 may be configured so that simultaneous cooperation between the magnetic elements 1704A-1704I of ablation component 1700 and the magnetic elements of ablation component 1750 may result in a clamping force distributed along at least a portion of the length of the jaws 1702, 1752. For example, the ablation components 1700, 1750 may be configured to exert a substantially uniform pressure along the lengths of the jaws 1702, 1752 in connection with creating a generally linear ablation. For example, the structures illustrated in FIG. 17 may be configured to create a generally C-shaped linear lesion. Some examples may include faces that are substantially flat. Alternatively, some examples may include faces having non-planar shapes.

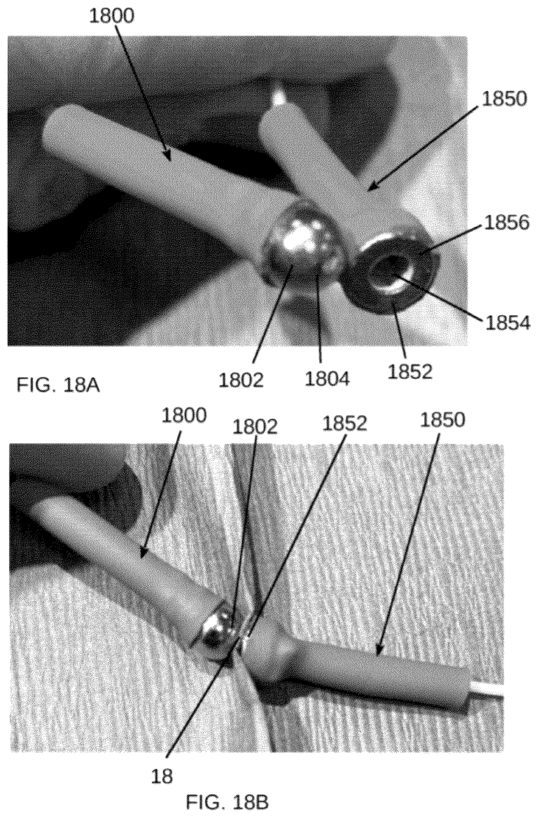

FIGS. 18A and 18B are perspective view examples of ablation components 1800, 1850, all according to at least some aspects of the present disclosure. Ablation components 1800, 1850 may be generally similar to other ablation components described herein and repeated description is omitted for brevity. In this example, ablation components 1800, 1850 may comprise selectively engageable cooperating surface features. For example, ablation component 1800 may include a distal end portion 1802 configured to mechanically cooperate with distal end portion 1852 of ablation component 1850 with the target tissue (e.g., heart wall 18) therebetween. For example, the distal end portion 1802 of ablation component 1800 may include a projection 1804, which may be generally rounded and/or at least partially bulbous or include an otherwise atraumatic shape. The distal end portion 1852 of ablation component 1850 may include a recess 1854 configured to engage at least a portion of the projection 1804. In this example, the recess 1854 may be at least partially defined by a peripheral wall 1856, which may be generally in the form of a ring. It is within the scope of the disclosure to utilize generally concave and/or convex cooperating surface features comprising any other shapes, such as polygons, ovals, etc. In some alternative examples, cooperating surface features may comprise elongated, generally parallel elements, such as generally parallel rails and recesses.

### Electromagnet Coupled Ablation Components

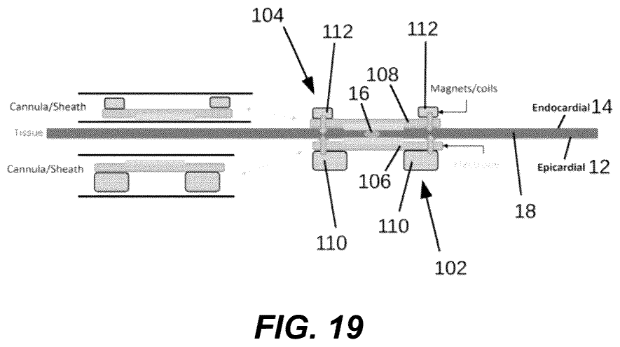

FIG. 19 is simplified schematic view of an example ablation system 100 in use on a heart 10, according to at least some aspects of the present disclosure. With continued reference to FIG. 1, as well as FIG. 19, the example ablation system 100 may include a first ablation component 102 configured to be positioned on a first side of a target tissue and/or a second ablation component 104 configured to be positioned on a second side of the target tissue. The first and second sides of the target tissue may be opposite sides. For example, the first ablation component 102 may be configured to be positioned on an epicardial (exterior) surface 12 of the heart 10. The second ablation component 104 may be configured to be positioned on an endocardial (interior) surface 14 of the heart 10.

The first ablation component 102 may include at least one ablation element 106, such as an electrode, and the second ablation component 104 may include at least one ablation element 108, such as an electrode. The ablation elements 106, 108 may be configured to cooperate to create a lesion 16 in the target tissue, such as a transmural lesion extending through the full thickness of the heart wall 18.

The first ablation component 102 may include at least one magnetic element 110 and the second ablation component 104 may include at least one magnetic element 112. The magnetic elements 110, 112 may be configured to cooperate to facilitate positioning the first ablation component 102 and/or the second ablation component 104. For example, the magnetic elements 110, 112 may be utilized to align the ablation elements 106, 108 across the heart wall 18 to create the lesion 16 at the desired location. Some examples may utilize selective coupling and/or uncoupling of the magnetic elements 110, 112 to facilitate positioning the electrodes as well as for subsequent repositioning to nearby target tissues for subsequent ablations. Some example ablation components 102, 104 may be configured to at least partially compress tissue therebetween. For example, the magnetic elements 110, 112 may be configured to cooperate to compress the target tissue (e.g., heart wall 18) between the ablation components 102, 104, which may improve ablation outcomes in some circumstances. Some examples may be configured to control the damping force, such as to provide sufficient, but not excessive, compression of the target tissue.

The example ablation system 100 may include a control unit 114. The control unit 114 may be operable to control the operation of the ablation components 102, 104. In some examples, the control unit 114 may be configured to selectively provide ablation energy (e.g., RF energy from an electrosurgical generator) to electrodes comprising the ablation elements 106, 108. In some examples, the control unit 114 may be configured to control the operation of the magnetic elements 110, 112. For example, the control unit 114 may be configured to selectively apply electrical current through one or more electromagnets comprising one or more of the magnetic elements 110, 112. The ablation components 102, 104 may include respective connecting elements 116, 118, which may operatively couple the ablation elements 106, 108 and/or the magnetic elements 110, 112 to the control unit 114. The connecting elements 116, 118 may include or be used in connection with one or more electrical conductors, fluidic conduits, mechanical force transmission components, catheters, wires, steerable portions, sheaths, trocars, hemostatic components, etc.

In some examples, the ablation system 100 may be configured to access the target tissue (e.g., heart wall 18) via a surgical approach (open-chest and/or minimally invasive) and/or via the patient's vascular system. For example, the first ablation component 102 may be positioned on the epicardial surface 12 via a percutaneous or surgical sub-xiphoid or intercostal access or incision 120, such as through the chest wall 20. The second ablation component 104 may be positioned on the endocardial surface 14 via a blood vessel 22 (e.g., the femoral vein). In various circumstances, the routes used to obtain access to the epicardial and endocardial surfaces may be determined based at least in part upon factors such as the portion of the heart wall 18 that comprises the target tissue for the ablation, the patient's potential anatomical variations, previous surgeries, etc. In some examples, either or both of the first ablation component 102 or the second ablation component 104 may access the target tissue by any suitable route, such as open chest, thoracotomy, sub-xiphoid, trans-diaphragmatic, and/or vascular access.

In some examples, one or more of the ablation components 102, 104 may include and/or may be configured to operate in connection with a device configured to selectively and/or releasably attach to the target tissue, such as a tissue anchor. For example, the first ablation component 102 may include a vacuum anchor 122. Which may be configured to selectively and/or releasably secure the first ablation component 102 to the epicardial surface 12. In some such examples, the connecting element 116 may include a vacuum line and/or the control unit 114 may include a vacuum pump and/or related control apparatus.

Generally, some examples may be operated as follows. Appropriate access to the epicardial 12 and endocardial 14 surfaces may be obtained. The first ablation component 102 may positioned at a desired location on the epicardial surface 12. The magnetic elements 110, 112 may be utilized (e.g., magnetically coupled) to position and/or secure the second ablation component 104 on the endocardial surface 14. For example, the magnetic clement 110 of the first ablation component 102 may magnetically attract the magnetic element 112 of the second ablation component 104. The ablation elements 106, 108 may be operated to ablate the target tissue and create the lesion 16. The magnetic elements 110, 112 may be uncoupled. The first and second ablation components 102, 104 may be withdrawn from the patient.

Some examples may provide sensing capabilities for various parameters. For example, tissue impedance may be sensed between ablation electrodes, such as to assess the extent of an ablation. In some examples, measurements of magnetic fields may be utilized, such as to determine a distance between certain components.

The ablation elements 106, 108 and magnetic elements 110, 112 are illustrated as separate elements in FIGS. 1 and 19 by way of example, however, in some other examples, one or more of the ablation elements 106, 108 and/or one or more of the magnetic elements 110, 112 may be combined into a single element. For example, at least a portion of a magnetic element 110, 112 may also act as an ablation element 106, 108 (e.g., an RF electrode), or a dedicated electrode material may be used around or adjacent to a magnetic element 110, 112.

Various examples according to at least some aspects of the present disclosure may include one or more magnetic elements. For example, each ablation component may comprise one or more magnetic elements. Magnetic elements may comprise, for example, any combination of one or more permanent magnets, electromagnets, and/or ferromagnetic materials. Individual magnetic elements mentioned above may comprise one or more constituent subcomponents, which may be shaped, arranged, configured, combined, and/or controlled to achieve desired magnetic and/or mechanical characteristics. In some examples, electromagnets may be energized (with constant or varying electrical current) to attract and/or repel other magnetic elements, such as by reversing current direction to reverse magnetic polarity and/or varying current flow to adjust magnetic field strength. For example, the current supplied to an electromagnet may be constant direct current (DC) and/or may be pulsed to change the attractive and/or repulsive forces. In some examples, magnetic elements (such as permanent magnets) may be repositioned (e.g., translated and/or reoriented) to vary the arrangement of the magnetic field. In some drawings and descriptions herein, magnetic poles of various magnetic elements may be described as north (“N”) and south (“S”), however, it should be understood that these descriptions are illustrative and generally represent opposite polarities. Generally, opposite polarities (N-S or S-N) attract each other, and identical polarities (S-S or N-N) repel each other.

Magnetic elements and associated technologies according to at least some aspects of the present disclosure, as well as any feature, characteristic, or methodology relating thereto, may be used in connection with any medical or surgical device. For example, magnetic elements and associated technologies according to at least some aspects of the present disclosure may be utilized in connection with any ablation system (or component thereof) employing magnetic elements and/or magnetic coupling of ablation components. In various examples described herein, repeated description of the ablation or other components, elements, or systems may be omitted for brevity. However. it will be understood that any magnetic element described herein may be utilized in connection with any ablation or other element, component, or system according to the present disclosure and/or may be paired or combined with any other magnetic element according to the present disclosure.

The present disclosure contemplates that magnetic elements utilized in ablation systems may be subject to certain design considerations, such as the magnitudes and directions of forces exerted by the magnetic elements and the dimensions and package sizes of the magnetic elements. For example, some ablation components may be configured to contact a target tissue following delivery through a cannula, catheter, or similar device. Some example ablation components, such as those used endocardially, may be delivered through a catheter having an internal diameter of about 5 mm or less. Some example ablation components, such as those used epicardially, may be delivered through a cannula having an internal diameter of about 10 mm or less. Some example ablation components may have a longitudinal length of about 20-30 mm, excluding the connecting elements, though the foregoing exemplary dimensions may be exceeded or reduced in certain instances.

The present disclosure contemplates that the force between two magnetic elements may be given by the following equation, in which F is the force, m₁ is the magnetic moment of a first magnetic element, m₂ is the magnetic moment of a second magnetic element, x is the center-to-center distance between the first magnetic element and the second magnetic element, and μ₀ is the free space permeability:

$$F = \frac{3 {μ}_{0} {m}_{1} {m}_{2}}{2 π {x}^{4}}$$

The force may be increased by, for example, increasing the moment of the first magnetic element and/or the second magnetic element, ensuring collinear alignment of the magnetic elements, and/or reducing the distance between the magnetic elements,

The present disclosure contemplates that the magnetic moment of a ferromagnet may be given by the following equation, in which m is the magnetic moment, M is the magnetization, V is the volume of the magnetic element, Bᵣ is the flux density, and μ₀ is the free space permeability:

$$m = MV = \frac{{B}_{r} V}{{μ}_{0}}$$

The magnetic moment may be increased by, for example, increasing the volume of the magnetic element and/or increasing the flux density by utilizing a different material.

The present disclosure contemplates that the flux density of some example materials may differ as indicated in the table below:

| | Bᵣ |
|---|---|
| | Tesla |
| Material | (T) |
| SᵣFe₁₂O₁₉ | 0.42 |
| Alnico 5 | 1.25 |
| SmCo5 | 0.88 |
| Sm₂Co₁₇ | 1.08 |
| Nd₂Fe₁₄B | 1.28 |

The present disclosure contemplates that an electromagnet generally comprises a core wrapped with a wire (e.g., a coil) through which electrical current is directed. The magnetic moment of an electromagnetic coil with a soft magnetic core may be given by the following equation, in which m the magnetic moment, μ_{eff} is the effective permeability of the core, N is the number of turns of the wire in the coil, I is the current through the wire, and A is the coil loop area:

$$m = {μ}_{eff} NIA$$

The effective permeability of the core may depend on the core materials, relative permeability and/or the core shape. Generally, it is within the scope of the disclosure to optimize characteristics of electromagnets (e.g., shape, coil wire cross section, winding geometry, and/or materials (e.g., washers)) to increase current density in the coil, which may increase the magnetic moment of an electromagnet.

The present disclosure contemplates that the more stray flux a shape has, the harder it may be to align the internal moments, thus producing a low μ_{eff}. Generally, long and thin magnetic elements may have relatively low stray flux. They may be a low energy shape and may have a relatively higher effective permeability. Generally, short and wide magnetic elements may have relatively high stray flux. They may be a high energy shape and may have a relatively lower effective permeability.

The present disclosure contemplates that a generally U-shaped magnetic element may be referred to as a “horseshoe magnet.” Generally, horseshoe magnetic elements may reduce stray flux and may increase effective permeability as compared to other shapes.

The descriptions herein of various example magnetic elements focuses on the differences between various example configurations. Description of aspects or features common among several examples may not be repeated for brevity. Accordingly, it should be understood that various aspects, features, and characteristics described in connection with any example may also pertain to other examples, whether or not explicitly noted.

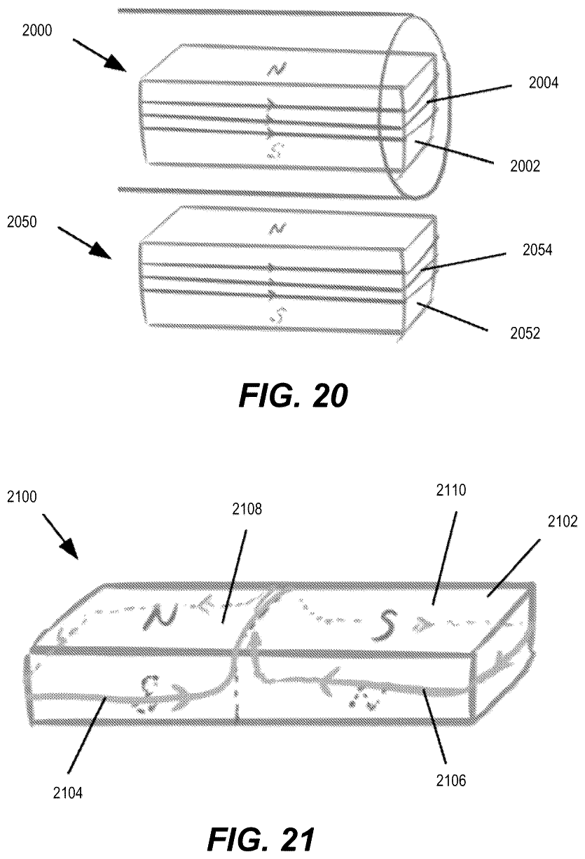

FIG. 20 is a simplified perspective view of example magnetic elements 2000, 2050, according to at least some aspects of the present disclosure. Magnetic elements 2000, 2050 may each comprise a core 2002, 2052, which may be generally in the form of a cuboid, such as a large rectangular cuboid. Windings (e.g., coils) 2004, 2054 may be disposed at least partially around the cores 2002, 2052 to form electromagnets. For example, the coils 2004, 2054 may be wound around the long edges of the cores 2002, 2052. In some examples, the cores may be relatively large to maximize the coil area.

FIG. 21 is a simplified perspective view of an example magnetic element 2100, according to at least some aspects of the present disclosure. Magnetic element 2100 may include a core, such as a large rectangular cuboid core 2102. Magnetic element 2100 may include a plurality of coils 2104, 2106. In some examples, coils 2104, 2106 may have opposite winding directions (e.g., they may be counter wound). For example, coil 2104 may be left-hand wound and/or coil 2106 may be right-hand wound. In some circumstances, utilizing counter-wound coils 2104, 2106 may facilitate locating and positioning operations with cooperating magnetic elements of other ablation components. In some examples, utilizing counter-wound coils 2104, 2106 may form two, oppositely oriented (e.g., north-south/south-north) electromagnet portions (e.g., lobes) 2108, 2110 using respective portions of the core 2102.

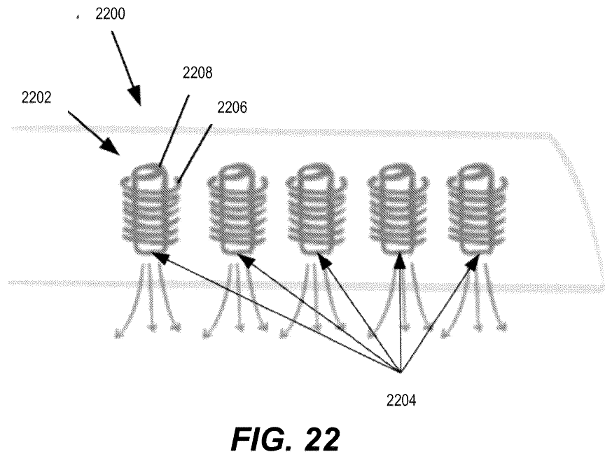

FIG. 22 is a simplified perspective view of an example magnetic element 2200, according to at least some aspects of the present disclosure. Magnetic element 2200 may include an array 2202 comprising a plurality of relatively small electromagnets 2204, each comprising a respective coil 2206 wound about a respective core 2208. In some examples, the array 2202 may provide advantages over a single coil wound about a single core having a similar size as the array 2202. In some examples, each individual core 2208 may have a generally high aspect ratio. That is, an individual core 2208 may have a relatively high length to diameter (L/d) ratio. Some alternative examples may comprise a similar arrangement (e.g., an array) of permanent magnets in place of the electromagnets 2204.

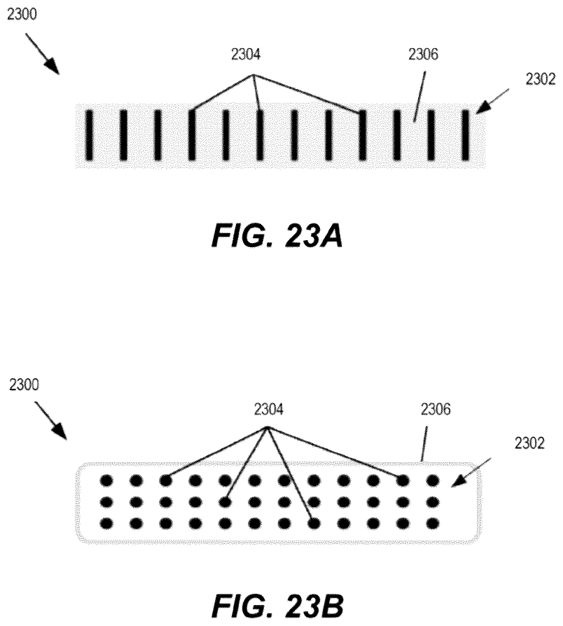

FIG. 23A is a simplified elevation view of an example magnetic element 2300 and FIG. 23B is a simplified plan view of the example magnetic element 2300, all according to at least some aspects of the present disclosure. Magnetic element 2300 may comprise an electromagnet including an array 2302 comprising a plurality of relatively small cores 2304, which may be ferrous. In some examples, the cores 2304 may have relatively high length to diameter (L/d) ratios. Magnetic element 2300 may include a coil 2306, such as a copper coil, disposed generally around the array 2302. In some examples, the relatively large coil 2306 may provide a field that may be focused by the cores 2304.

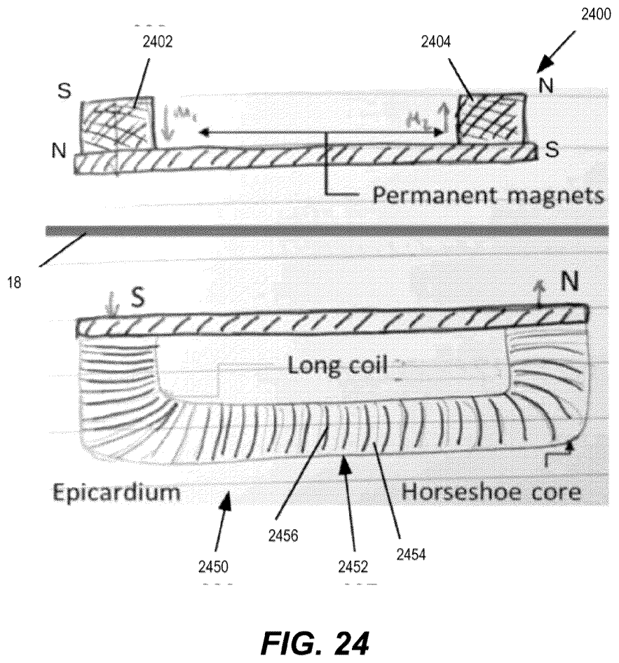

FIG. 24 is a simplified cross-sectional view of example cooperating magnetic elements 2400, 2450, according to at least some aspects of the present disclosure. Magnetic element 2400 may comprise permanent magnets 2402, 2404, which may be arranged with opposite polarities. Magnetic element 2450 may comprise an electromagnet, such as a horseshoe electromagnet 2452. Electromagnet 2452 may include a generally horseshoe-shaped core 2454 wound with a coil 2456, which may be relatively long and/or have a relatively large number of turns. In some examples, the horseshoe shape may be “long” and “squat” in shape. In some examples, the magnetic polarity of the electromagnet 2452 and the permanent magnets 2402, 2404 may be configured to allow the electromagnet 2452 and the permanent magnets 2402, 2404 to cooperate (e.g., magnetically attract each other) to clamp the target tissue (e.g., heart wall 18). For example, the north pole of the permanent magnet 2402 may be oriented towards the south pole of the electromagnet 2452 and/or the south pole of the permanent magnet 2404 may be oriented towards the north pole of the electromagnet 2452.

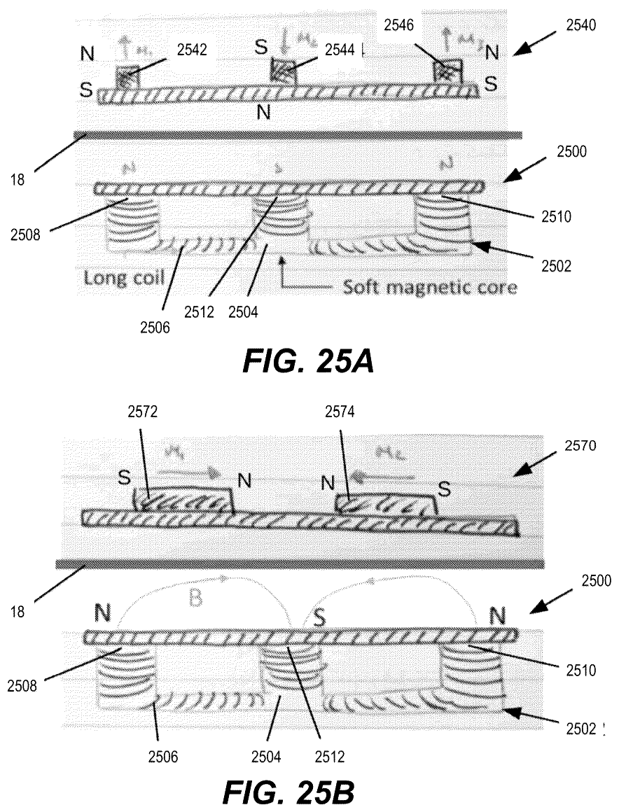

FIGS. 25A and 25B are simplified cross-sectional views of example cooperating magnetic elements 2500, 2540, 2570, all according to at least some aspects of the present disclosure.

Magnetic element 2500 may comprise a double horseshoe electromagnet 2502. The double horseshoe magnet 2502 may include a double horseshoe (e.g., generally E-shaped) core 2504 wound with a coil 2506. As illustrated in FIGS. 25A and 25B, the double horseshoe electromagnet 2502 provides two north poles 2508, 2510 and one south pole 2512 therebetween. Alternative configurations (e.g., reversed electrical current flow or windings) may provide a similar arrangement, but with two south poles and one north pole therebetween.

Magnetic element 2540 (FIG. 25A) may comprise permanent magnets 2542, 2544, 2546 positioned to cooperate with individual respective poles 2508, 2512, 2510 of the double horseshoe electromagnet 2502, such as to facilitate magnetic attraction of the magnetic elements 2500, 2540 to compress the target tissue (e.g., heart wall 18) therebetween. For example, the south pole of permanent magnet 2542 may be arranged to face the north pole 2508 of the double horseshoe electromagnet, the north pole of permanent magnet 2544 may be arranged to face south pole 2512, and/or the south pole of permanent magnet 2546 may be arranged to face north pole 2510.

Magnetic element 2570 (FIG. 25B) may comprise a permanent magnet 2572 positioned to cooperate with the poles 2508, 2512 of the double horseshoe magnet 2502. For example, permanent magnet 2572 may be positioned generally longitudinally between poles 2508, 2512 and/or may be arranged so that its poles are opposite polarities of the corresponding poles 2508, 2512 of the double horseshoe magnet 2502. Magnetic element 2570 may comprise a permanent magnet 2574 positioned to cooperate with poles 2510, 2512 of the double horseshoe magnet 2502. For example, permanent magnet 2574 may be positioned generally longitudinally between poles 2510, 2512 and/or may be arranged so that its poles are opposite polarities of the corresponding poles 2510, 2512 of the double horseshoe magnet 2502.

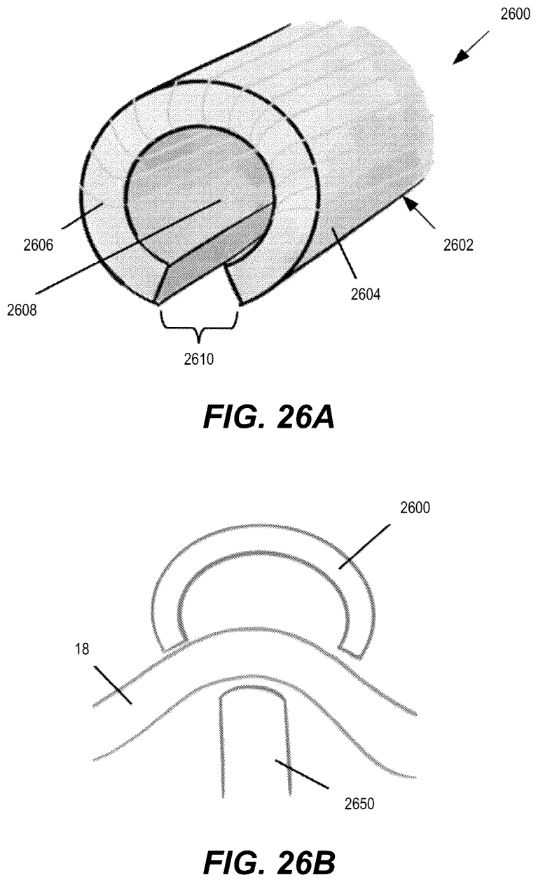

FIG. 26A is a simplified perspective view of an example magnetic element 2600 and FIG. 26B is a simplified cross-sectional view, of exemplary cooperating magnetic elements 2600, 2650, all according to at least some aspects of the present disclosure. Magnetic element 2600 may include an electromagnet 2602 generally in the form of a segment of a tube, such as an angular segment of a generally circular cylindrical tube. For example, the core 2604 may be generally in the form of an angular segment of a generally circular cylindrical tube, and the coils 2606 may be wound thereabout. In some examples, the electromagnet 2602 may include a recess 2608 having an opening 2610, such as the elongated, generally longitudinally oriented opening illustrated in FIG. 26A. In some examples, the electromagnet 2602 may be formed generally as a horseshoe utilizing at least a portion of the circumference of a cannula. Magnetic element 2650 may be configured to cooperate with magnetic element 2600 across the target tissue (e.g., heart wall 18), such as is illustrated in FIG. 26B. In this example, the magnetic element 2650 may be sized and/or shaped to cooperated with the opening 2610 of the magnetic element 2600 so that the heart tissue 18 is pressed at least partially into the opening 2610. For example, the magnetic clement 2650 may have a generally elongated shape corresponding to the opening 2610 and/or may be magnetically attracted to the magnetic element 2600 to position the target tissue 18 as shown in FIG. 26B. Some examples may utilize the flexibility of the target tissue 18 to facilitate positioning of the tissue. In various alternative examples, electromagnets, permanent magnets, and/or ferromagnetic materials may be utilized in each of the magnetic elements 2600, 2650.

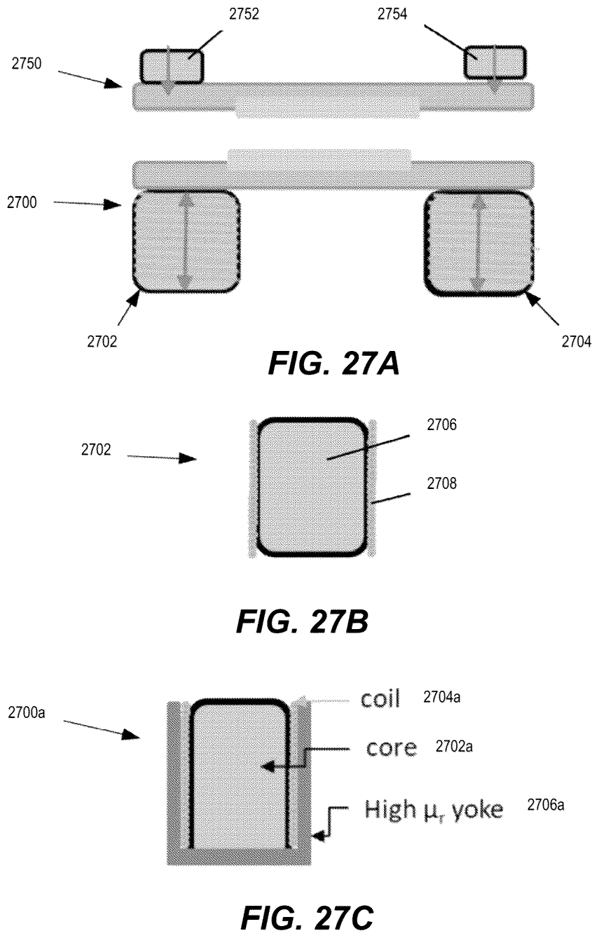

FIG. 27A is a simplified cross-sectional view of exemplary cooperating magnetic elements 2700, 2750, according to at least some aspects of the present disclosure. FIG. 27B is a simplified cross-sectional view of example electromagnet 2702, according to at least some aspects of the present disclosure. FIG. 27C is a simplified cross-sectional view of an alternative example electromagnet 2700a, according to at least some aspects of the present disclosure. Referring to FIG. 27A, an example magnetic element 2750 may comprise two permanent magnets 2752, 2754 similar to that described elsewhere herein. As illustrated in FIG. 27B, some example electromagnets 2702, 2704 may comprise a core 2706 and a winding 2708. FIG. 27C illustrates an alternative electromagnet 2700A, which may be used in place of an electromagnet 2702, 2704, for example. Electromagnet 2700A may comprise a core 2702A and a coil 2704A. Additionally, electromagnet 2700A may comprise a yoke 2706A disposed about at least a portion of the core 2702A and/or the coil 2704A. The yoke 2706A may be constructed from one or more materials having a relatively high magnetic permeability (μᵣ). In some examples, including a yoke may reduce unused stray flux and/or may increase the effective permeability of the core (μ_{eff}).

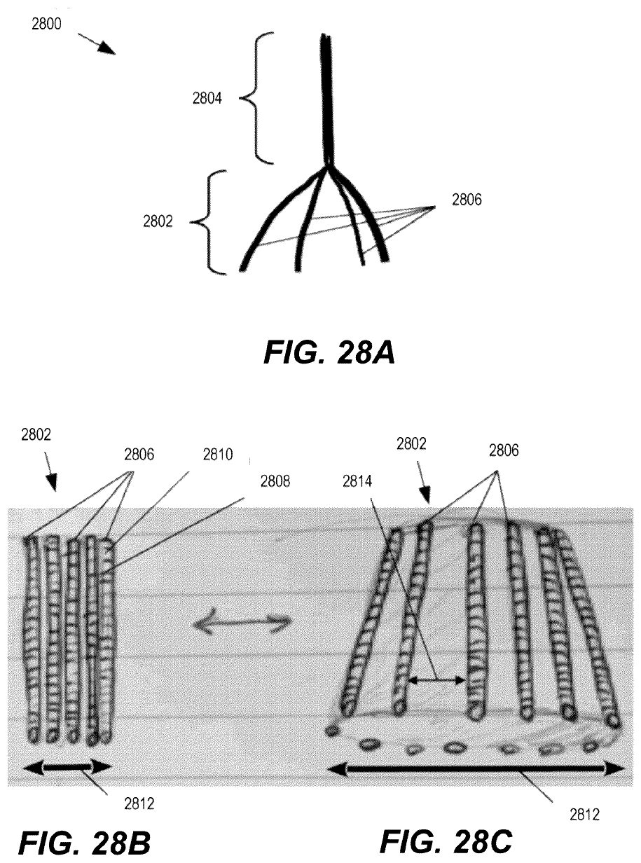

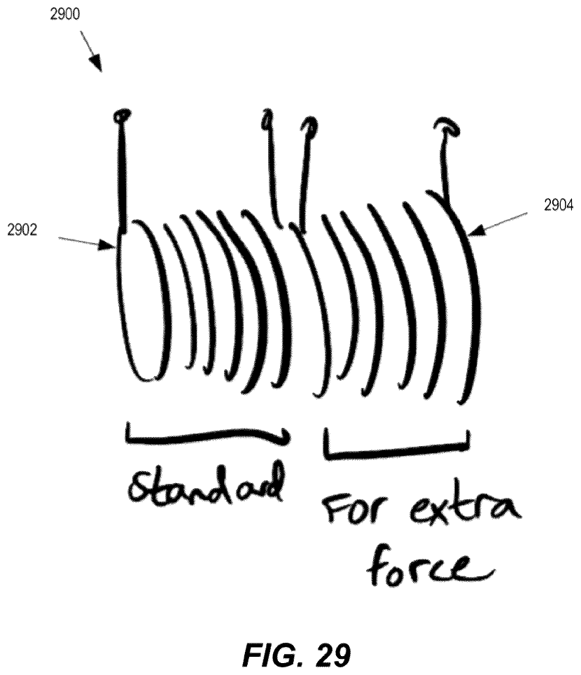

FIG. 28A is a simplified elevation view of an example expandable magnetic element 2800 in an expanded configuration and FIGS. 28B and 28C are detailed elevation views of an example expandable portion 2802 of the magnetic element 2800 in retracted and expanded configurations, respectively, all according to at least some aspects of the present disclosure. Referring to FIG. 28A, the magnetic element 2800 may comprise an expandable portion 2802 and/or a non-expandable portion 2804. The expandable portion 2802 may be disposed distally on the non-expandable portion 2804. The expandable portion 2802 may comprise a plurality of relatively long and thin electromagnets 2806, which may be arranged generally longitudinally. Referring to FIGS. 28B and 28C, each electromagnet 2806 may comprise a coil 2808 disposed about a core 2810. The expandable portion 2802 may be reconfigurable between a retracted configuration (FIG. 28B) and an expanded configuration (FIG. 28C). In the expanded configuration, a lateral width 2812 of the expanded portion (e.g., the diameter) may be substantially greater than in the retracted configuration. In the expanded configuration, the distance 2814 between adjacent electromagnets may be substantially greater than in the retracted configuration.

FIG. 29 is a simplified schematic view of an example magnetic element 2900, according to at least some aspects of the present disclosure. Magnetic element 2900 comprises two or more coils 2902, 2904. In some examples, the coils 2902, 2904 may be separately energized and/or controlled, such as by the control unit 114 (see FIG. 1). For example, the first coil 2902 may be energized for some operations (while the second coil 2904 remains deenergized), and both the first coil 2902 and the second coil 2904 may be energized for other operations. In some examples, energizing both the first coil 2902 and the second coil 2904 may produce a stronger magnetic field from the magnetic element 2900 than energizing only one of the coils 2902, 2904. Accordingly, one or both of the coils 2902, 2904 may be energized to produce the desired magnetic field strength and, therefore, the force applied by the magnetic element 2900. As will be appreciated by those of skill in the art, whether the respective magnetic fields caused by the coils 2902, 2904 are additive or subtractive in a particular circumstance may depend upon the direction of the windings comprising the coils 2902, 2904 and/or the direction of the current flow in each coil 2902, 2904. Generally, some example magnetic elements may comprise a plurality of electrically separate coils, which may be separately energized to increase or decrease the magnetic fields and/or resulting forces exerted by the magnetic elements.

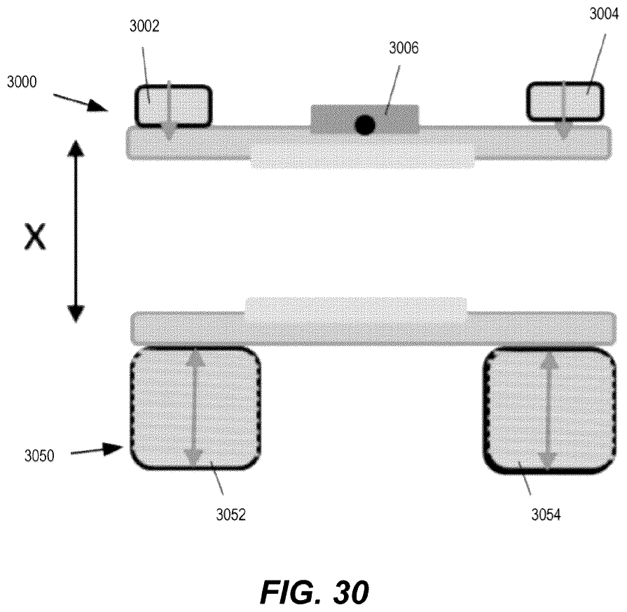

FIG. 30 is a simplified cross-sectional view of example magnetic elements 3000, 3050, according to at least some aspects of the present disclosure. Magnetic element 3000 may comprise one or more permanent magnets 3002, 3004 and/or magnetic element 3050 may comprise one or more electromagnets 3052, 3054, similar to that described elsewhere herein. Magnetic element 3000 may include a sensor 3006 configured to detect and/or provide a signal associated with the detected magnetic field strength. For example, the sensor 3006 may provide a signal associated with the detected strength of the magnetic field(s) associated with the electromagnets 3052, 3054 of the magnetic element 3050. The resulting signal may be used to estimate the force applied to a target tissue between the magnetic elements 3000, 3050 and/or to estimate a distance X between the magnetic elements 3000, 3050. In some examples, the resulting signal may be used to adjust the current applied to electromagnets, such as to control the clamping force applied by the magnetic elements.

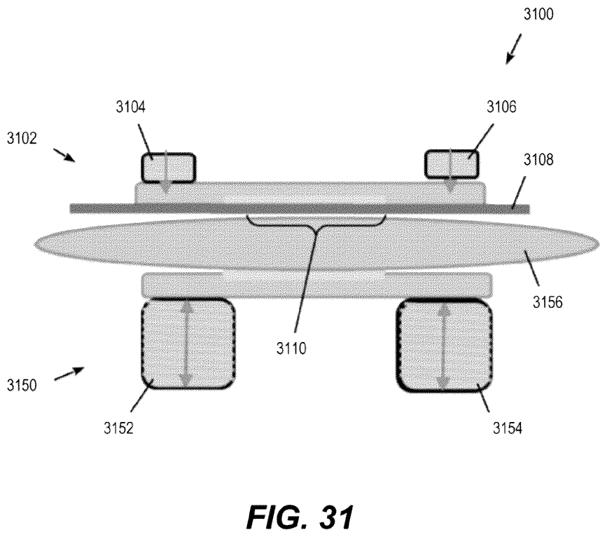

FIG. 31 is a simplified cross-sectional view of an example ablation system 3100, according to at least some aspects of the present disclosure. Ablation system 3100 may include a first ablation component 3102 and a second ablation component 3150. The ablation components may include magnetic elements 3104, 3106, 3152, 3154 configured to cooperate to exert a clamping force on a target tissue 3108. Ablation component 3150 may include an expandable element 3156, which may comprise an inflatable or inflated element, such as a balloon, mechanically interposing the magnetic elements 3152, 3154 and the target tissue 3108. Generally, the expandable clement 3156 may be configured so that the contact area 3110 between the expandable element 3156 and the target tissue 3108 increases as clamping force increases. As a result, the pressure exerted on the target tissue 3108 by the second ablation component 3150 may increase with increased clamping force at a slower rate than if the second ablation component 3150 did not include an expandable portion 3156.

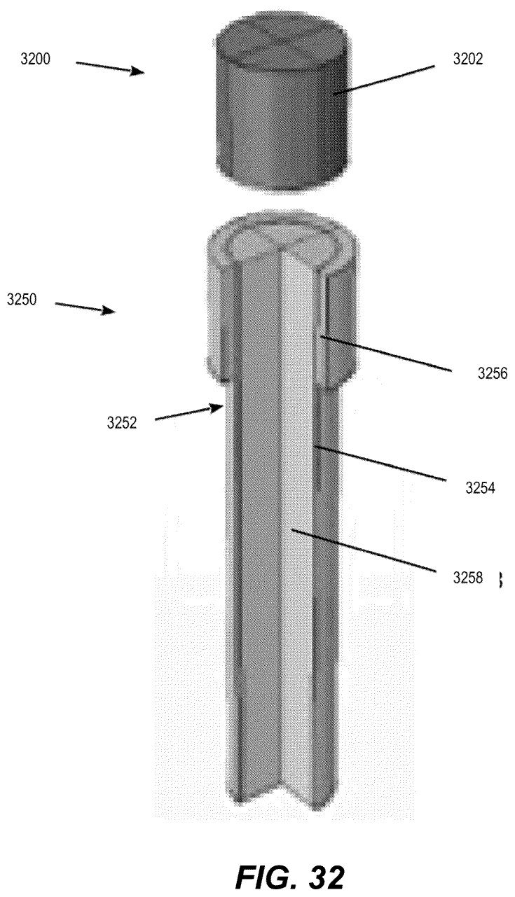

FIG. 32 is a simplified perspective section view of exemplary cooperating magnetic elements 3200, 3250, according to at least some aspects of the present disclosure. Magnetic element 3200 may comprise an axially magnetized cylindrical permanent magnet 3202.

Magnetic element 3250 may comprise an axially oriented electromagnet 3252, which may include two or more coils 3254, 3256 disposed about an axially oriented, generally cylindrical core 3258. In some examples, the coils 3254, 3256 may be at least partially concentrically disposed about the core 3258. In some examples, the inner coil 3254 may be disposed about the core 3258 for most or all of the axial length of the core 3258 and/or the outer coil 3256 may be disposed concentrically about the inner core 3254 for substantially less than the axial length of the core 3258. In an alternative example, the inner coil 3254 may be shorter than the outer coil 3256 and/or the outer coil 3256 may extend for most or all of the axial length of the core 3258.

In some examples, the coils 3254, 3256 may be separately energized and/or controlled, such as by the control unit 114 (see FIG. 1). For example, the inner coil 3254 may be energized for some operations (while the outer coil 3256 remains deenergized), and both the inner coil 3254 and the outer coil 3256 may be energized for other operations. In some examples, energizing both the inner coil 3254 and the outer coil 3256 may produce a stronger magnetic field from the magnetic element 3250 than energizing only one of the coils 3254, 3256. Accordingly, one or both of the coils 3254, 3256 may be energized to produce the desired magnetic field strength and, therefore, the force applied by the magnetic element 3250. As will be appreciated by those of skill in the art, whether the respective magnetic fields caused by the coils 3254, 3256 are additive or subtractive in a particular circumstance may depend upon the direction of the windings comprising the coils 3254, 3256 and/or the direction of the current flow in each coil 3254, 3256. Generally, some example magnetic elements may comprise a plurality of electrically separate coils, which may be separately energized to increase or decrease the magnetic fields and/or resulting forces exerted by the magnetic elements.

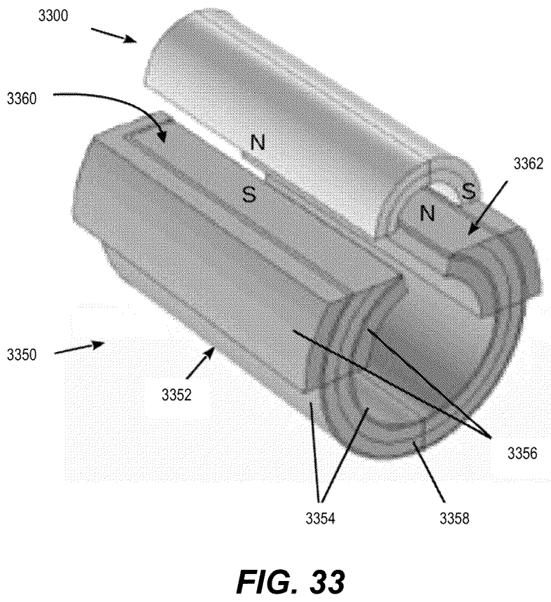

FIG. 33 is a simplified perspective view of example cooperating magnetic elements 3300, 3350, according to at least some aspects of the present disclosure. A magnetic element 3300 may comprise a permanent magnet generally in the form of an elongated angular section of a tube (e.g., a semi-cylindrical), such as a hollow right circular cylinder. Magnetic element 3300 may act as a horseshoe magnet, with opposite poles at opposite circumferential ends. Magnetic element 3350 may comprise electromagnet 3352 generally in the form of an elongated angular section of a tube, such as a hollow right circular cylinder. The electromagnet 3352 may include two or more coil 3354, 3356 disposed about a core 3358. In some examples, coils 3354, 3356 may be individually selectively energized similar to the examples described above with reference to FIGS. 29 and 32. In the example illustrated in FIG. 33, inner coil 3354 may be disposed about most or all of the inner and outer circumferential surfaces of the core 3358 and/or the outer coil 3356 may be disposed about the inner coil 3354 for less than all of the inner and outer circumferential surfaces of the core 3358. Alternative examples may include other relative sizes of coils 3354, 3356, similar to those described above with reference to FIG. 32.

Magnetic element 3350 may act as a horseshoe magnet, with opposite poles at opposite circumferential ends. In some examples, the core 3358 may include generally flattened tissue facing surfaces 3360, 3362, which may be arranged generally opposite corresponding generally flat surfaces of magnetic element 3300. In some alternative examples, the magnetic element 3300 may be replaced by a diametrically magnetized cylindrical permanent magnet, such as one having the form of a right circular cylinder.

Although some examples are described herein with reference to RF ablation, it is within the scope of the disclosure to utilize alternative and/or additional ablation modalities in various alternative examples according to at least some aspects of the present disclosure. Namely, examples may utilize pulsed field ablation, cryoablation, ultrasound ablation, and/or laser ablation and may include ablation elements appropriately configured for such modalities in addition to or in place of electrodes described herein.

Various examples according to at least some aspects of the present disclosure may include one or more magnetic elements. Magnetic elements may comprise, for example, any combination of one or more permanent magnets, electromagnets, and/or ferromagnetic materials. Individual magnetic elements mentioned herein may comprise one or more magnetic subcomponents, which may be shaped, arranged, configured, combined, and/or controlled to achieve desired magnetic and/or mechanical characteristics. In some examples, electromagnets may be energized (with constant or varying electrical current) to attract and/or repel other magnetic elements, such as by reversing current direction to reverse magnetic polarity and/or varying current flow to adjust magnetic field strength. In some examples, magnetic elements (such as permanent magnets) may be repositioned (e.g., translated and/or reoriented) to vary the arrangement of a given or varied magnetic field.

Generally, any ablation component described herein may be utilized epicardially and/or endocardially, such as in cooperation with any other ablation component described herein. Examples may utilize magnetic coupling between magnetic elements associated with each ablation component. In some examples, if a magnetic element of the one ablation component comprises a permanent magnet, then a magnetic element of the other ablation component may comprise a ferromagnetic material, a permanent magnet, and/or an electromagnet. In some examples, if a magnetic element of one ablation component comprises an electromagnet, then a magnetic element of the other ablation component may comprise a ferromagnetic material, a permanent magnet, and/or an electromagnet.

Some examples may be configured to utilize substantially constant magnetic coupling forces. Some examples may be configured to vary force with separation distance between magnetic elements. For example, the distance between magnetic elements may be measured by imaging, measured by distance sensors (e.g., magnetic field sensors), and/or by measured by initial impedance.

Some examples may be configured to vary forces in connection with ablation cycles. For example, force may be increased and/or decreased as the ablation elements get closer (e.g., separation distance decreases). In some examples, forces may be increased and/or decreased as impedance through tissue changes. In some examples, magnetic polarity may be reversed (e.g., from an attractive arrangement to a repulsive arrangement), such as when it may be desired to disengage one or both of the ablation components from the tissue. For example, the direction of current flow through an electromagnet may be reversed and/or a movable permanent magnet may be rotated.

Some examples may be configured to facilitate user driven force changes. For example, some magnetic elements (e.g., permanent magnets) may rotatable to facilitate disengagement from tissue. Some examples may provide the ability for a user to selectively shield a magnetic element to increase and/or decrease forces. Some examples may provide the ability for a user to move a magnetic element closer to and/or farther from a cooperating magnetic element to increase and/or decrease forces.

In some examples, power delivery to ablation elements, such as for RF ablation, may be impedance controlled. In some examples, power delivery based on impedance may be independent of magnetic forces, magnetic forces may be used to modify an impedance calculation, and/or magnetic forces may be used to assess contact before and/or during an ablation.

In some examples, power delivery to ablation elements, such as for RF ablation, may be power controlled. In some examples, the power control may be substantially independent of magnetic forces, magnetic forces may be used to adjust power, magnetic forces may be used to modify a voltage calculation, and/or magnetic forces may be used to assess contact before and/or during ablation.

In some examples, energy delivery to ablation elements, such as for RF ablation, may be continuous, pulsed, and/or switched.

The following patent references are hereby incorporated by reference in their entireties: US20050187545A1, WO2005081868A3, WO2005081868A2, US20070239155A1, U.S. 8,419,729B2, US20110152860A1, U.S. Pat. Nos. 9,072,522B2, 9,173,705B2, US20110313286A1, US20120123411A1, U.S. Pat. No. 9,474,574B2, US20130172864A1, U.S. Pat. No. 9,101,364B2, 9,445,864B2, US20150366605A1, US20160242750A1, U.S. Pat. No. 10,610,208B2, WO2016138008A1, US20160242788A1, EP3261514A1, EP3261514A4, U.S. Pat. No. 10,548,611B2, US20170354457A1, U.S. Pat. No. 10,398,495B2, US20170258520A1, U.S. Pat. No. 10,251,699B2, US20190247113A1, US20200000511A1, US20200261096A1, and US20200205797A1.

Example methods of ablating tissue and/or creating a lesion may include operations involving use of various examples described herein. Example methods of manufacturing ablation apparatus and components thereof may include operations associated with acquiring, producing, and assembling various parts, elements, components, and systems described herein.

Unless specifically indicated, it will be understood that the description of the structure, function, and/or methodology with respect to any illustrative example herein may apply to any other illustrative example. More generally, it is within the scope of the present disclosure to utilize any one or more features of any one or more examples described herein in connection with any other one or more features of any other one or more other examples described herein. Accordingly, any combination of any of the features or examples described herein is within the scope of this disclosure.

The terminology used herein is for the purpose of describing particular examples only and is not intended to be limiting of the invention. As used herein, the singular forms “a”, “an” and “the” are intended to include the plural forms as well, unless the context clearly indicates otherwise. It will be further understood that the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present innovation has been presented for purposes of illustration and description but is not intended to be exhaustive or limited to the innovation in the form disclosed. Many modifications and variations will be apparent to those of ordinary skill in the art without departing from the scope and spirit of the innovation. The examples were described in order to explain the principles of the innovation and the practical application, and to enable others of ordinary skill in the art to understand the innovation, including various alternate examples incorporating various modifications as are suited to the particular use contemplated.

Following from the above description and invention summaries, it should be apparent to those of ordinary skill in the art that, while the methods and apparatuses herein described constitute examples according to the present disclosure, it is to be understood that the scope of the disclosure contained herein is not limited to the above precise examples and that changes may be made without departing from the scope of the disclosure. Likewise, it is to be understood that it is not necessary to meet any or all of the identified advantages or objects disclosed herein in order to fall within the scope of the disclosure, since inherent and/or unforeseen advantages may exist even though they may not have been explicitly discussed herein.

What is claimed is:

1. An ablation system comprising:
a first ablation component configured to be positioned on a first side of a target tissue, the first ablation component comprising a first ablation element and a first magnetic element;
a second ablation component configured to be positioned on a second side of a target tissue, the second side being opposite the first side, the second ablation component comprising a second ablation element and a second magnetic element;
wherein the first magnetic element and the second magnetic element are configured to cooperate to facilitate positioning at least one of the first ablation component and the second component on the target tissue; and
wherein the first ablation element and the second ablation element are configured to cooperate to create a lesion in the target tissue.

2.-7. (canceled)

8. A method of creating a lesion, the method comprising:
placing a first ablation component on a first surface of a target tissue, the first ablation component comprising a first magnetic element and a first ablation element;
placing a second ablation component on a second surface of a target tissue, the second ablation component comprising a second magnetic element and a second ablation element;
positioning one or more of the first ablation component or the second ablation component using magnetic cooperation between the first magnetic element and the second magnetic element; and
creating a first lesion in the target tissue using the first ablation element and the second ablation element.

9. The method of claim 8, wherein one or more of the first magnetic element or the second magnetic element comprises an electromagnet; and
wherein positioning the one or more of the first ablation component or the second ablation component using magnetic cooperation between the first magnetic element and the second magnetic element comprises energizing the electromagnet.

10. The method of claim 8, wherein creating the lesion in the target tissue using the first ablation element and the second ablation element comprises one or more of radiofrequency (RF) energy ablation, pulsed field ablation, cryoablation, ultrasound ablation, or laser ablation.

11. The method of claim 8, further comprising
repositioning the one or more of the first ablation component or the second ablation component; and
creating a second lesion in the target tissue proximate the first lesion via at least one of simultaneous monopolar ablation and bipolar ablation.

12. The method of claim 8, further comprising, securing one or more of the first ablation component or the second ablation component to the target tissue using a tissue anchor.

13. An ablation system comprising:
a first ablation component configured to be positioned on a first side of a target tissue, the first ablation component comprising a first magnetic element;
a second ablation component configured to be positioned on a second side of a target tissue, the second side being opposite the first side, the second ablation component comprising a second magnetic element;
wherein the first magnetic element and the second magnetic element are configured to cooperate to facilitate positioning at least one of the first ablation component and the second ablation component on the target tissue.

14.-19. (canceled)

20. A method of creating a lesion, the method comprising:
placing a first ablation component on a first surface of a target tissue, the first ablation component comprising a first ablation magnetic element;
placing a second ablation component on a second surface of a target tissue, the second ablation component comprising a second ablation magnetic element;
aligning and compressing the target tissue between the first and second ablation magnetic elements using magnetic attraction; and
creating a first lesion in the target tissue using at least one of the first ablation magnetic element and the second ablation magnetic element.

21. The method of claim 20, wherein:
at least one of the first ablation magnetic element and the second ablation magnetic element comprises an electromagnet; and
aligning and compressing the target tissue between the first and second ablation magnetic elements using magnetic attraction includes energizing the electromagnet.

22. The method of claim 20, wherein creating the first lesion in the target tissue using at least one of the first ablation magnetic element and the second ablation magnetic element includes using at least one of radiofrequency (RF) energy ablation, pulsed field ablation, cryoablation, ultrasound ablation, and laser ablation.

23. The method of claim 20, further comprising
repositioning at least one of the first ablation magnetic component and the second ablation magnetic component; and
creating a second lesion in the target tissue proximate the first lesion.

24. The method of claim 20, further comprising, securing at least one of the first ablation magnetic component and the second ablation magnetic component to the target tissue using a tissue anchor.

25. An ablation device comprising:
a first ablation component configured to be positioned on a first side of a target tissue, the first ablation component comprising a first ablation element and a first magnetic element;
a first catheter operatively coupled to the first ablation component; and,
a second catheter having a through conduit sized to receive the first ablation component and the first catheter.

26. The ablation device of claim 25, wherein the first ablation element and the first magnetic element are synonymous.

27. The ablation device of claim 25, wherein the ablation device further includes an atraumatic distal tip.

28. The ablation device of claim 27, wherein the atraumatic distal tip comprises at least one of the first ablation element and the first magnetic element.

29.-81. (canceled)

82. A method of monitoring a tissue compression procedure, the method comprising:
positioning a first medical device and a second medical device so as to be interposed by tissue, the first medical device including a magnet, the second medical device including at least one of a magnet and a magnetically attractive material, where at least one of the first medical device and the second medical device includes a magnetic sensor;
obtaining outputs from the magnetic sensor while the medical devices are interposed by the tissue;
estimating at least one of a distance between the first medical device and the second medical device, and a compression force applied to the tissue by at least one of the first medical device and the second medical device.

83. An ablation device comprising:
a first ablation component configured to be positioned on a first side of a target tissue, the first ablation component comprising a first ablation element and a first magnetic element;
a first tether operatively coupled to the first ablation component; and,
a catheter having a through conduit sized to receive the first ablation component and the tether.

84.-88. (canceled)
